SEQUENCE LISTING (28 sequences)

SEQ ID NO: 1 - protein, 119 aa, Homo sapiens
  B2M (UniProt P61769-1, v1)
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 2 - protein, 99 aa, Homo sapiens [sequence omitted]

SEQ ID NO: 3 - protein, 89 aa, Homo sapiens
  B2M Ig-like C1-type domain (residues 25-113 of UniProt P61769-1, v1)
PKIQVYSRHPAENGKSNFLNCYVSGFHPSDIEVDLLKNGERIEKVEHSDLSFSKDWSFYLLYYTEFTPTEKDEYACRVNHVTLSQPKIV

SEQ ID NO: 4 - protein, 119 aa, Macaca mulatta
  B2M (UniProt Q6V7J5-1, v1)
MSRSVALAVLALLSLSGLEAIQRTPKIQVYSRHPPENGKPNFLNCYVSGFHPSDIEVDLLKNGEKMGKVEHSDLSFSKDWSFYLLYYTEFTPNEKDEYACRVNHVTLSGPRTVKWDRDM

SEQ ID NO: 5 - protein, 28 aa, Artificial Sequence
  Description of Artificial Sequence Synthetic peptide
  CD8alpha transmembrane domain
IYIWAPLAGTCGVLLLSLVITLYCNHRN

SEQ ID NO: 6 - protein, 26 aa, Artificial Sequence
  Description of Artificial Sequence Synthetic peptide
  CD28 transmembrane domain
WVLVVVGGVLACYSLLVTVAFIIFWV

SEQ ID NO: 7 - protein, 4 aa, Artificial Sequence
  Description of Artificial Sequence Synthetic peptide
  ITAM motif
  MOD_RES (2)..(3): Any naturally occurring amino acid
  MOD_RES (4)..(4): L or I
YXXX

SEQ ID NO: 8 - protein, 16 aa, Artificial Sequence
  Description of Artificial Sequence Synthetic peptide
  ITAM motif
  MOD_RES (2)..(3): Any naturally occurring amino acid
  MOD_RES (4)..(4): L or I
  MOD_RES (5)..(12): Any naturally occurring amino acid
  MISC_FEATURE (5)..(12): This region may encompass 6-8 residues
  MOD_RES (14)..(15): Any naturally occurring amino acid
  MOD_RES (16)..(16): L or I
YXXXXXXXXXXXYXXX

SEQ ID NO: 9 - protein, 112 aa, Artificial Sequence
  Description of Artificial Sequence Synthetic polypeptide
  CD3-zeta intracellular domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 41 aa, Artificial Sequence
  Description of Artificial Sequence Synthetic polypeptide
  CD28 intracellular domain
RSKRSRLLHSDYMNMTPRRPGPTRKHYQPYAPPRDFAAYRS

SEQ ID NO: 11 - protein, 13 aa, Homo sapiens
  Spacer region (Human HLA-A*02)
LRWEPSSQPTIPI

SEQ ID NO: 12 - protein, 20 aa, Homo sapiens
  Signal peptide(Human B2M)
MSRSVALAVLALLSLSGLEA

SEQ ID NO: 13 - protein, 4 aa, Artificial Sequence
  Description of Artificial Sequence Synthetic peptide
  2A autocleavage linker sequence
NPGP

SEQ ID NO: 14 - protein, 22 aa, Artificial Sequence
  Description of Artificial Sequence Synthetic peptide
  2A autocleavage linker sequence
GSGATNFSLLKQAGDVEENPGP

SEQ ID NO: 15 - protein, 138 aa, Artificial Sequence
  Description of Artificial Sequence Synthetic polypeptide
  Q8 marker
MGLVRRGARAGPRMPRGWTALCLLSLLPSGFMAELPTQGTFSNVSTNVSPAKPTTTPAPRPPTPAPTIASQPLSLRPEACRPAAGGAVHTRGLDFACDIYIWAPLAGTCGVLLLSLVITLYCNHRNRRRVCKCPRPVV

SEQ ID NO: 16 - DNA, 357 bp, Homo sapiens
  B2M coding sequence
atgagcagatctgtggccctggctgtgctggccctgctgtctctgtctggcctggaagccatccagcggacccccaagatccaggtgtacagcagacaccccgccgagaacggcaagagcaacttcctgaactgctacgtgtccggcttccaccccagcgacatcgaggtggacctgctgaagaacggcgagcggatcgagaaggtggaacacagcgacctgagcttcagcaaggactggtccttctacctgctgtactacaccgagttcacccccaccgagaaggacgagtacgcctgcagagtgaaccacgtgaccctgagccagcccaagatcgtgaagtgggaccgggacatg

SEQ ID NO: 17 - DNA, 39 bp, Homo sapiens
  Spacer region (Human HLA-A*02) coding sequence
ctgagatgggagcccagcagccagcctaccatccccatc

SEQ ID NO: 18 - DNA, 84 bp, Artificial Sequence
  Description of Artificial Sequence Synthetic oligonucleotide
  CD8alpha transmembrane domain coding sequence
atctacatctgggcccctctggccggcacctgtggcgtgctgctgctgagtctcgtgatcaccctgtactgcaaccaccggaac

SEQ ID NO: 19 - DNA, 336 bp, Artificial Sequence
  Description of Artificial Sequence Synthetic polynucleotide
  CD3-zeta intracellular domain coding sequence
cgagtgaagttttccagaagtgcggacgctcctgcctaccaacaaggccagaaccaactgtacaatgagttaaacttaggaaggagggaagagtacgacgtgctagacaagcgccgtggaagagatcctgaaatgggcggcaagcccagacggaagaacccccaggaaggcctgtataacgaactgcagaaagacaagatggccgaggcctacagcgagatcggcatgaagggcgaaagaaggcggggcaagggccacgatggcctgtatcagggactgagcaccgccaccaaggacacctacgacgccctgcacatgcaggctctgcctccgcgg

SEQ ID NO: 20 - DNA, 78 bp, Artificial Sequence
  Description of Artificial Sequence Synthetic oligonucleotide
  CD28 transmembrane domain coding sequence
tgggtgctggttgtggtgggaggggtactggcctgctatagcttactggtgactgttgcctttattattttttgggtt

SEQ ID NO: 21 - DNA, 123 bp, Artificial Sequence
  Description of Artificial Sequence Synthetic polynucleotide
  CD28 intracellular domain coding sequence
cgaagtaagcgttcccggctgctgcacagtgactacatgaatatgaccccaagacggcccggaccgacaaggaaacactatcaaccctatgctcccccacgagactttgctgcctacagatca

SEQ ID NO: 22 - DNA, 66 bp, Artificial Sequence
  Description of Artificial Sequence Synthetic oligonucleotide
  2A autocleavage linker coding sequence
ggaagcggcgccacaaatttcagcctgctgaagcaggccggcgacgtggaagagaatcccggccct

SEQ ID NO: 23 - DNA, 414 bp, Artificial Sequence
  Description of Artificial Sequence Synthetic polynucleotide
  Q8 marker coding sequence
atgggactcgtgcgcagaggcgctagagccggccctagaatgcctagaggatggaccgccctgtgcctgctgtctctgctgcctagcggcttcatggccgagctgcctactcagggcaccttcagcaacgtgtccaccaatgtgtccccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtc

SEQ ID NO: 24 - protein, 480 aa, Artificial Sequence
  Description of Artificial Sequence Synthetic polypeptide
  cFLIP (long isoform)
MSAEVIHQVEEALDTDEKEMLLFLCRDVAIDVVPPNVRDLLDILRERGKLSVGDLAELLYRVRRFDLLKRILKMDRKAVETHLLRNPHLVSDYRVLMAEIGEDLDKSDVSSLIFLMKDYMGRGKISKEKSFLDLVVELEKLNLVAPDQLDLLEKCLKNIHRIDLKTRIQKYKQSVQGAGTSYRNVLQAAIQKSLKDPSNNFRLHNGRSKEQRLKEQLGAQQEPVKKSIQESEAFLPQSIPEERYKMKSKPLGICLIIDCIGNETELLRDTFTSLGYEVQKFLHLSMHGISQILGQFACMPEHRDYDSFVCVLVSRGGSQSVYGVDQTHSGLPLHHIRRMFMGDSCPYLAGKPKMFFIQNYVVSEGQLEDSSLLEVDGPAMKNVEFKAQKRGLCTVHREADFFWSLCTADMSLLEQSHSSPSLYLQCLSQKLRQERKRPLLDLHIELNGYMYDWNSRVSAKEKYYVWLQHTLRKKLILSYT

SEQ ID NO: 25 - protein, 376 aa, Artificial Sequence
  Description of Artificial Sequence Synthetic polypeptide
  PI-9
METLSNASGTFAIRLLKILCQDNPSHNVFCSPVSISSALAMVLLGAKGNTATQMAQALSLNTEEDIHRAFQSLLTEVNKAGTQYLLRTANRLFGEKTCQFLSTFKESCLQFYHAELKELSFIRAAEESRKHINTWVSKKTEGKIEELLPGSSIDAETRLVLVNAIYFKGKWNEPFDETYTREMPFKINQEEQRPVQMMYQEATFKLAHVGEVRAQLLELPYARKELSLLVLLPDDGVELSTVEKSLTFEKLTAWTKPDCMKSTEVEVLLPKFKLQEDYDMESVLRHLGIVDAFQQGKADLSAMSAERDLCLSKFVHKSFVEVNEEGTEAAAASSCFVVAESSMESGPRFCADHPFLFFIRHNRANSILFCGRFSSP

SEQ ID NO: 26 - DNA, 1440 bp, Artificial Sequence
  Description of Artificial Sequence Synthetic polynucleotide
  cFLIP (long isoform) coding sequence
atgagcgccgaagtgatccaccaggtggaagaggccctggacaccgacgagaaagagatgctgctgttcctgtgccgcgacgtggccattgatgtggtgcctccaaatgtgcgggacctgctggacatcctgagagagcggggaaaactgagcgtgggagatctggccgagctgctgtatagagtgcggagattcgacctgctgaagcggatcctgaagatggaccggaaggccgtggaaacccatctgctgagaaaccctcacctggtgtccgactacagagtgctgatggccgagatcggcgaggacctggataagtccgatgtgtccagcctgatcttcctgatgaaggactacatgggcagaggcaagatcagcaaagagaagtccttcctggacctggtggtggaactggaaaagctgaacctggtggcccctgaccagctggatctgctggaaaagtgcctgaagaacatccaccggatcgacctgaaaacccggatccaaaagtacaagcagagcgtgcaaggcgccggaaccagctacagaaatgtgctgcaggccgccatccagaagtccctgaaggaccccagcaacaacttccggctgcacaacggcagaagcaaagagcagcggctgaaagaacagctgggagcccagcaagagcccgtgaagaagtccatccaagagagcgaggcattcctgcctcagagcatccctgaggaacggtacaagatgaagtccaagcctctgggcatctgcctgatcatcgactgcatcggcaacgagacagagctgctgagagacacctttaccagcctgggctacgaggtgcagaagttcctgcatctgagcatgcacggcatcagccagatcctgggccagttcgcctgtatgcccgagcacagagactacgacagcttcgtgtgtgtgctggtgtctagaggcggcagccagtctgtgtacggcgtggaccaaacacactctggcctgcctctgcaccacattcggagaatgttcatgggcgacagctgcccttatctggccggcaagcccaagatgttctttatccaaaactacgtcgtgtccgagggacagctggaagatagcagcctgctggaagtggatggccctgccatgaagaacgtggaattcaaggcccagaaacggggcctgtgtaccgtgcacagagaggccgatttcttctggtcactgtgcaccgccgacatgtcactgctggaacagagccactctagccccagcctgtacctgcagtgtctgagccagaagctgcggcaagagagaaagaggcccctgctcgacctgcacatcgagctgaacggctatatgtacgactggaacagccgggtgtccgccaaagaaaagtactacgtgtggctgcagcataccctgcggaagaagctgatcctgtcctacacc

SEQ ID NO: 27 - DNA, 1128 bp, Artificial Sequence
  Description of Artificial Sequence Synthetic polynucleotide
  PI-9 coding sequence
atggaaaccctgagcaatgccagcggcaccttcgccatcagactgctgaagatcctgtgccaggacaaccccagccacaacgtgttctgtagccccgtgtctatcagcagcgccctggctatggttctgctgggcgccaagggaaataccgccacacagatggctcaggccctgtctctgaacaccgaagaggacatccaccgggcctttcagagcctgctgaccgaagtgaacaaggccggcacacagtacctgctgagaaccgccaatcggctgttcggcgagaaaacctgccagttcctgagcaccttcaaagagagctgcctgcagttctaccacgccgagctgaaagagctgagcttcatcagagccgccgaggaaagccggaagcacatcaatacctgggtgtccaagaaaaccgagggcaagatcgaggaactgctgcccggcagctccatcgatgccgaaacaagactggtgctggtcaacgccatctacttcaaaggcaagtggaacgagcccttcgacgagacatacaccagagagatgcccttcaagatcaatcaagaggaacagcggcccgtgcagatgatgtaccaagaggccaccttcaagctggcccatgtgggagaagttcgggcccaactgctggaactgccctacgccagaaaagaactgtccctgctggtgctgctgcctgacgatggcgtggaactgagcaccgtggaaaagagcctgaccttcgagaagctgaccgcctggaccaagcctgactgcatgaagtccaccgaggtggaagtgctgctccccaagttcaagctgcaagaggactacgacatggaaagcgtgctgcggcacctgggaatcgtggatgctttccagcagggcaaagccgacctgtctgccatgtctgccgagagggatctgtgcctgagcaagttcgtgcacaagagcttcgtggaagtgaacgaggaaggcacagaagccgccgctgccagctcttgttttgtggtggccgagagcagcatggaatctggccctagattctgcgccgaccatccttttctgttcttcatccggcacaaccgggccaacagcatcctgttctgtggcagattcagcagcccc

SEQ ID NO: 28 - protein, 6 aa, Artificial Sequence
  Description of Artificial Sequence Synthetic 6xHis tag
HHHHHH

(19) United States
(12) Patent Application Publication
(10) Pub. No.: US 2021/0238255 A1
(43) Pub. Date: Aug. 5, 2021

(54) CHIMERIC HLA ACCESSORY RECEPTOR

(71) Applicant: Baylor College of Medicine, Houston, TX (US)

(72) Inventors: David Quach, Houston, TX (US); Cliona M. Rooney, Houston, TX (US)

(73) Assignee: Baylor College of Medicine, Houston, TX (US)

(21) Appl. No.: 17/050,289

(22) PCT Filed: Apr. 18, 2019
(86) PCT No.: PCT/US2019/028201

§ 371 Date: Oct. 23, 2020

Related U.S. Application Data
(60) Provisional application No. 62/745,341, filed on Oct. 13, 2018.
(60) Provisional application No. 62/661,339, filed on Apr. 23, 2018.

(51) Int. Cl. C07K 14/74; C07K 14/725; C07K 14/705; C12N 5/0783; A61P 37/06; A61K 35/17

(52) U.S. Cl. CPC C07K 14/70539; C07K 14/7051; C07K 14/70517; C07K 14/70521; C12N 5/0636; A61P 37/06; A61K 38/00; C07K 2319/03; C07K 2319/02; C12N 2502/99; C12N 2502/11; C12N 2510/00; A61K 35/17

(57) ABSTRACT

Polypeptides comprising: (i) an MHC class I α polypeptide association domain, (ii) a transmembrane domain, and (iii) a signalling domain comprising an ITAM-containing sequence are disclosed. Also disclosed are nucleic acids and expression vectors encoding, compositions comprising, and methods using such polypeptides.

This application claims priority from U.S. 62/661,339 filed 23 Apr. 2018, and U.S. 62/745,341 filed 13 Oct. 2018, the contents and elements of which are herein incorporated by reference for all purposes.

### TECHNICAL FIELD

The present disclosure relates at least to the fields of molecular biology, cell biology, immunology, cell therapy, and medicine. The present disclosure also relates to methods of medical treatment and prophylaxis.

### BACKGROUND

In solid organ transplants (SOT) or hematopoietic stem cell transplants (HSCT), mismatches in HLA between recipient and donor can lead to rejection of the organ or graft-vs-host disease (GVHD), respectively. Immunosuppressive drugs can mitigate these outcomes, but due to their broadly inhibitory action against immune cells, they increase the risk of opportunistic infections. Alloreactive T cells that recognise mismatched HLA via their TCR are major mediators of graft rejection and GVHD.

### SUMMARY

In a first aspect, the present disclosure provides a polypeptide, optionally isolated, comprising: (i) at least one MHC class I α polypeptide association domain, (ii) at least one transmembrane domain, and (iii) at least one signalling domain comprising at least one immunoreceptor tyrosine-based activation motif (ITAM)-comprising sequence.

In some embodiments the MHC class I α polypeptide association domain comprises an amino acid sequence which is, or which is derived from, the Ig-like C1-type domain of B2M.

In some embodiments the MHC class I α polypeptide association domain comprises an amino acid sequence having at least 70% amino acid sequence identity to SEQ ID NO:3.

In some embodiments the signalling domain comprises an amino acid sequence which is, or which is derived from, the intracellular domain of CD3-.

In some embodiments the signalling domain comprises an amino acid sequence having at least 70% amino acid sequence identity to SEQ ID NO:9.

In some embodiments the transmembrane domain comprises an amino acid sequence which is, or which is derived from, the transmembrane domain of CD8a or CD28.

In some embodiments the transmembrane domain comprises an amino acid sequence having at least 70% amino acid sequence identity to SEQ ID NO:5 or SEQ ID NO:6.

In some embodiments the signalling domain additionally comprises at least one costimulatory sequence.

In some embodiments the costimulatory sequence is, or is derived from, the intracellular domain of CD28.

In some embodiments the signalling domain comprises an amino acid sequence having at least 70% amino acid sequence identity to SEQ ID NO:10.

In some embodiments the polypeptide additionally comprises a spacer region between the MHC class I α polypeptide association domain and the transmembrane domain.

In some embodiments the spacer region comprises an amino acid sequence having at least 70% amino acid sequence identity to SEQ ID NO:11.

The present disclosure also provides a nucleic acid, or a plurality of nucleic acids, optionally isolated, encoding a polypeptide described herein.

In some embodiments the nucleic acid or plurality of nucleic acids comprise at least one control element for inducible upregulation of expression of the polypeptide.

In some embodiments the nucleic acid or plurality of nucleic acids comprise at least one control element for tissue-specific expression of the polypeptide.

In some embodiments the nucleic acid or plurality of nucleic acids encodes a conditional expression system for controlling expression of the polypeptide.

In some embodiments the conditional expression system for controlling expression of the polypeptide is a Tet-On system.

The present disclosure also provides an expression vector, or a plurality of expression vectors, comprising a nucleic acid or a plurality of nucleic acids described herein.

The present disclosure also provides a cell comprising a polypeptide, a nucleic acid or plurality of nucleic acids, or an expression vector or plurality of expression vectors described herein.

In some embodiments the cell is a virus-specific T cell or plurality thereof.

The present disclosure also provides a method comprising culturing a cell comprising a nucleic acid or a plurality of nucleic acids or an expression vector or a plurality of expression vectors described herein, under conditions suitable for expression of the polypeptide from the nucleic acid(s) or expression vector(s).

The present disclosure also provides a method of generating or expanding a population of immune cells, comprising modifying an immune cell of any kind to express or comprise a polypeptide, a nucleic acid or plurality of nucleic acids, or an expression vector or plurality of expression vectors described herein.

The present disclosure also provides a method of generating or expanding a population of immune cells, comprising:
(a) isolating immune cells from a subject;
(b) modifying at least one immune cell to express or comprise a polypeptide, a nucleic acid or plurality of nucleic acids, or an expression vector or plurality of expression vectors described herein; and
(c) optionally expanding the modified at least one immune cell.

The present disclosure also provides a method of generating or expanding a population of virus-specific immune cells, comprising:
(a) isolating immune cells from a subject;
(b) generating or expanding a population of virus-specific immune cells by a method comprising: stimulating the immune cells by culture in the presence of antigen presenting cells (APCs) presenting a peptide of the virus;
(c) modifying at least one virus-specific immune cell to express or comprise a polypeptide, a nucleic acid or plurality of nucleic acids, or an expression vector or plurality of expression vectors described herein; and
(d) optionally expanding the modified at least one virus-specific immune cell.

The present disclosure also provides a cell obtained or obtainable by a method described herein, and/or a plurality thereof.

In some embodiments in accordance with various aspects of the present disclosure, the cell(s) additionally comprises modification to increase expression/activity of one or more factors capable of inhibiting apoptosis.

The present disclosure also provides a composition comprising a polypeptide, a nucleic acid or a plurality of nucleic acids, an expression vector or a plurality of expression vectors, or a cell described herein.

The present disclosure also provides a polypeptide, a nucleic acid or a plurality of nucleic acids, an expression vector or a plurality of expression vectors, a cell, or a composition described herein for use in a method of medical treatment or prophylaxis. The present disclosure provides methods of treatment or prophylaxis for an individual in need thereof, said method comprising delivering to the individual an effective amount of a polypeptide or a plurality of polypeptides, a nucleic acid or a plurality of nucleic acids, an expression vector or a plurality of expression vectors, a cell, or a composition described herein.

The present disclosure also provides a method of depleting a population of immune cells of alloreactive immune cells, comprising:
(a) modifying at least one immune cell from a first subject to express or comprise a polypeptide, a nucleic acid or plurality of nucleic acids, or an expression vector or plurality of expression vectors described herein; and
(b) contacting a population of immune cells to be depleted of alloreactive immune cells from a second, allogeneic subject with the modified at least one immune cell.

The present disclosure also provides a method of treating/preventing graft rejection following allotransplantation, comprising administering at least one immune cell of the donor subject for the allotransplant modified to express or comprise a polypeptide, a nucleic acid or plurality of nucleic acids, or an expression vector or plurality of expression vectors described herein to the recipient subject for the allotransplant.

The present disclosure also provides a method of treating/preventing graft versus host disease (GVHD) associated with allotransplantation, comprising contacting the allotransplant with at least one immune cell of the recipient subject for the allotransplant modified to express or comprise a polypeptide, a nucleic acid or plurality of nucleic acids, or an expression vector or plurality of expression vectors described herein.

The present disclosure also provides a method of treating/preventing a disease/condition by allotransplantation, comprising:
(a) modifying at least one immune cell from the donor subject to express or comprise a polypeptide, a nucleic acid or plurality of nucleic acids, or an expression vector or plurality of expression vectors described herein; and
(b) administering the modified at least one immune cell to the recipient subject for the allotransplant.

The present disclosure also provides a method of treating/preventing a disease/condition by allotransplantation, comprising:
(a) modifying at least one immune cell from the recipient subject for the allotransplant to express or comprise a polypeptide, a nucleic acid or plurality of nucleic acids, or an expression vector or plurality of expression vectors described herein; and
(b) contacting the allotransplant with the modified at least one immune cell.

In some embodiments the allotransplantation comprises adoptive transfer of allogeneic immune cells.

The present disclosure also provides a method of treating/preventing a disease/condition by adoptive transfer of allogeneic immune cells, comprising:
(a) isolating immune cells from a subject;
(b) modifying at least one immune cell to express or comprise a polypeptide, a nucleic acid or plurality of nucleic acids, or an expression vector or plurality of expression vectors described herein;
(c) optionally expanding the modified at least one immune cell, and;
(d) administering the modified at least one immune cell to a subject.

The present disclosure also provides a method of treating/preventing a disease/condition by adoptive transfer of allogeneic immune cells specific for a virus, comprising:
(a) isolating immune cells from a subject;
(b) generating or expanding a population of immune cells specific for a virus by a method comprising: stimulating the immune cells by culture in the presence of antigen presenting cells (APCs) presenting at least one peptide of the virus;
(c) modifying at least one immune cell specific for a virus to express or comprise a polypeptide, a nucleic acid or plurality of nucleic acids, or an expression vector or plurality of expression vectors described herein;
(d) optionally expanding the modified at least one immune cell specific for a virus, and;
(e) administering the modified at least one immune cell specific for a virus to a subject.

In some embodiments the immune cells are isolated from a first subject and administered to a second subject.

In some embodiments the disease/condition is a T cell dysfunctional disorder, a cancer or an infectious disease.

In some embodiments the cancer is selected from the group consisting of: colon cancer, colon carcinoma, colorectal cancer, nasopharyngeal carcinoma, cervical carcinoma, oropharyngeal carcinoma, gastric carcinoma, hepatocellular carcinoma, head and neck cancer, head and neck squamous cell carcinoma (HNSCC), oral cancer, laryngeal cancer, prostate cancer, lung cancer, small cell lung cancer, non-small cell lung cancer, bladder cancer, urothelial carcinoma, melanoma, advanced melanoma, renal cell carcinoma, ovarian cancer, mesothelioma, and a combination thereof.

The present disclosure also provides a method of depleting a population of immune cells of autoreactive immune cells, comprising:
(a) modifying at least one immune cell comprising/expressing an autoantigenic peptide:MHC class I α polypeptide complex to express or comprise a polypeptide, a nucleic acid or plurality of nucleic acids, or an expression vector or plurality of expression vectors described herein; and
(b) contacting a population of immune cells to be depleted of autoreactive immune cells (e.g. autoreactive T cells) with the modified at least one immune cell.

The present disclosure also provides a method of treating/preventing an autoimmune disease/condition in a subject, the method comprising administering to a subject an immune cell comprising/expressing: (i) an autoantigenic peptide:MHC class I α polypeptide complex and (ii) a polypeptide, a nucleic acid or plurality of nucleic acids, or an expression vector or plurality of expression vectors described herein.

### DESCRIPTION

The present disclosure relates to an approach for engineering therapeutic T cells to eliminate alloreactive T cells, which can contribute to allotransplant rejection. The present disclosure also relates to an approach for engineering therapeutic T cells to reduce the quantity of alloreactive T cells, which can contribute to allotransplant rejection, from a population of cells.

In a specific embodiment, a chimeric molecule was prepared that fuses β2 microglobulin (B2M)—a universal component of all MHC class I molecules—to the cytolytic domain of CD3ζ. This chimeric HLA Accessory Receptor (CHAR) is shown to be able to form a complex with endogenous HLA class I alpha chains via the B2M region within T cells expressing the CHAR. Binding of alloreactive T cells to the CHAR:HLA class I alpha complex triggers signalling through the CHAR and activation of the T cell expressing the CHAR, ultimately resulting in elimination of alloreactive T cells through effector function of the CHAR-expressing T cell.

The articles and methods of the present disclosure represent an improvement over prior art approaches to reduce/prevent destruction of allogeneic material by T cell-mediated alloreactive immune responses. Such approaches include e.g. treatment with one or more immunosuppressive agents, which is associated with increased susceptibility to infection amongst other side effects (see e.g. Khurana and Brennan, Current Concepts of Immunosuppression and Side Effects in H. Liapis and H. L. Wang (eds.), Pathology of Solid Organ Transplantation, 11 Springer-Verlag Berlin Heidelberg 2011). Another approach has been to inhibit/prevent HLA class I molecule expression by allogeneic cells/tissues, but this renders them susceptible to killing by NK cells.

### MHC Class I Variation

MHC class I molecules are non-covalent heterodimers of an alpha (α) chain and a beta (β)2-microglobulin (B2M). The α-chain has three domains designated α1, α2 and α3. The α1 and α2 domains together form the groove to which the peptide presented by the MHC class I molecule binds, to form the peptide:MHC complex. In humans, MHC class I α-chains are encoded by human leukocyte antigen (HLA) genes. There are three major HLA gene loci (HLA-A, HLA-B and HLA-C) and three minor loci (HLA-E, HLA-F and HLA-G).

MHC class I α-chains are polymorphic, and different α-chains are capable of binding and presenting different peptides. Genes encoding MHC class I α polypeptides are highly variable, with the result that cells from different subjects often express different MHC class I molecules.

This variability has implications for organ transplantation and adoptive transfer of cells between individuals. The immune system of a recipient of a transplant or adoptively transferred cells recognises the non-self MHC molecules as foreign, triggering an immune response directed against the transplant or adoptively transferred cells, which can lead to graft rejection. Alternatively, cells amongst the population of cells/tissue/organ to be transplanted may contain immune cells which recognise the recipient's MHC molecules as foreign, triggering an immune response directed against recipient tissues, which can lead to graft versus host disease (GVHD).

Alloreactive T cells comprise TCRs capable of recognising non-self MHC molecules (i.e. allogeneic MHC), and initiating an immune response thereto. Alloreactive T cells may display one or more of the following properties in response to a cell expressing a non-self MHC molecule: cell proliferation, growth factor (e.g. IL-2) expression, cytotoxic/effector factor (e.g. IFNγ, granzyme, perforin, granulysin, CD107a, TNFα, FASL) expression and/or cytotoxic activity.

“Alloreactivity” and an “alloreactive immune response” as used herein refers to an immune response directed against a cell/tissue/organ which is genetically non-identical to the effector immune cell. An effector immune cell may display alloreactivity or an alloreactive immune response to cells—or tissues/organs comprising cells—expressing non-self MHC/HLA molecules (i.e. MHC/HLA molecules which are non-identical to the MHC/HLA molecules encoded by the effector immune cells).

“MHC mismatched” and “HLA mismatched” subjects as referred to herein are subjects having MHC/HLA genes encoding non-identical MHC/HLA molecules. In some embodiments the MHC mismatched or HLA mismatched subjects have MHC/HLA genes encoding non-identical MHC class I α molecules. “MHC matched” and “HLA matched” subjects as referred to herein are subjects having MHC/HLA genes encoding identical MHC/HLA molecules. In some embodiments the MHC matched or HLA matched subjects have MHC/HLA genes encoding identical MHC class I α molecules.

Where a cell/tissue/organ is referred to herein as being allogeneic with respect to a reference subject/treatment, the cell/tissue/organ is from obtained/derived from cells/tissue/organ of a subject other than the reference subject and/or comprise MHC/HLA genes encoding MHC/HLA molecules (e.g. MHC class I α molecules) which are non-identical to the MHC/HLA molecules (e.g. MHC class I α molecules) encoded by the MHC/HLA genes of the reference subject. Where a cell/tissue/organ is referred to herein as being allogeneic with respect to a treatment, the cell/tissue/organ is from obtained/derived from cells/tissue/organ of a subject other than the subject to be treated, and/or comprise MHC/HLA genes encoding MHC/HLA molecules (e.g. MHC class I α molecules) which are non-identical to the MHC/HLA molecules (e.g. MHC class I α molecules) encoded by the MHC/HLA genes of the subject to be treated.

Where a cell/tissue/organ is referred to herein as being autologous with respect to a reference subject, the cell/tissue/organ is from obtained/derived from cells/tissue/organ of the reference subject. Where a cell/tissue/organ is referred to herein as being autogeneic with respect to a reference subject, cell/tissue/organ is genetically identical to the reference subject, or derived/obtained from a genetically identical subject. Where a cell/tissue/organ is referred to herein as being autologous in the context of a treatment of a subject (e.g. treatment by administration to a subject of autologous cells), the cell/tissue/organ is obtained/derived from cells/tissue/organ of the subject to be treated. Where a cell/tissue/organ is referred to herein as being autogeneic in the context of a treatment of a subject, the cell/tissue/organ is genetically identical to the subject to be treated, or derived/obtained from a genetically identical subject. Autologous and autogeneic cell/tissue/organs comprise MHC/HLA genes encoding MHC/HLA molecules (e.g. MHC class I α molecules) which are identical to the MHC/HLA molecules (e.g. MHC class I α molecules) encoded by the MHC/HLA genes of the reference subject.

Where a cell/tissue/organ is referred to herein as being allogeneic with respect to a reference subject, cell/tissue/organ is genetically non-identical to the reference subject, or derived/obtained from a genetically non-identical subject. Where a cell/tissue/organ is referred to herein as being allogeneic in the context of a treatment of a subject, the cell/tissue/organ is genetically non-identical to the subject to be treated, or derived/obtained from a genetically non-identical subject. Allogeneic cell/tissue/organs comprise MHC/HLA genes encoding MHC/HLA molecules (e.g. MHC class I α molecules) which are non-identical to the MHC/HLA molecules (e.g. MHC class I α molecules) encoded by the MHC/HLA genes of the reference subject.

### Polypeptides of the Disclosure

The present disclosure provides polypeptides comprising at least one MHC class I α polypeptide association domain, (ii) at least one transmembrane domain, and (iii) at least one signalling domain comprising an ITAM-containing sequence. Any polypeptides of the present disclosure may be non-natural, including synthetic. They may be recombinantly generated.

### MHC Class I α Polypeptide Association Domain

The polypeptide of the present disclosure comprises an MHC class I α polypeptide association domain, in specific embodiments. The MHC class I α polypeptide association domain is capable of associating with an MHC class I α polypeptide. In some embodiments, an MHC class I α polypeptide herein may be a polypeptide encoded by a HLA gene.

The polypeptide of the present disclosure is preferably capable of association with an MHC class I α polypeptide to form a complex (e.g. a heterodimer) comprising an MHC class I α polypeptide and the polypeptide of the disclosure. In particular embodiments the polypeptide of the disclosure and MHC class I α polypeptide are expressed by the same cell. In some embodiments the MHC class I α polypeptide association domain is capable of interacting with an MHC class I α polypeptide in the manner of B2M.

The MHC class I α polypeptide association domain of the polypeptide of the present disclosure provides for stable association (e.g. dimerisation) between the polypeptide of the disclosure and an MHC class I α polypeptide at the surface of a cell expressing the polypeptides. In some embodiments the MHC class I α polypeptide association domain provides for non-covalent association between the polypeptide of the disclosure and an MHC class I α polypeptide.

Human B2M polypeptide is translated as a 119 amino acid polypeptide having the amino acid sequence shown in SEQ ID NO:1 (UniProt: P61769-1, v1). After processing to remove the 20 amino acid signal peptide, mature B2M has the amino acid sequence shown in SEQ ID NO:2. B2M associates non-covalently with MHC class I α polypeptides through its Ig-like C1-type domain, which is shown in SEQ ID NO:3.

In some embodiments the MHC class I α polypeptide association domain of the polypeptide of the present disclosure comprises, or consists of, an amino acid sequence which is, or which is derived from, the Ig-like C1-type domain of B2M. In some embodiments the MHC class I α polypeptide association domain of the polypeptide of the present disclosure comprises, or consists of, an amino acid sequence which is, or which is derived from, the amino acid sequence of B2M.

Throughout this specification, an amino acid sequence which is “derived from” a given amino acid sequence may retain structural and/or functional properties of the amino acid sequence from which it is derived. The amino acid sequence may have high sequence identity to the amino acid sequence from which it is derived. For example, an amino acid sequence which is derived from a given sequence may have at least 80%, 85% 86%, 87%, 88%, 89%, 90%, 91%, 92%, 93%, 94%, 95%, 96%, 97%, 98%, 99%, or 100% sequence identity to the amino acid sequence from which it is derived. The amino acid sequence of a given protein or domain thereof can be retrieved from, or determined from a nucleic acid sequence retrieved from, databases known to the person skilled in the art. Such databases include UniProt, GenBank® and EMBL.

In this specification “B2M” refers to B2M from any species and includes B2M isoforms, fragments, variants (including mutants) or homologues from any species.

As used herein, a “fragment”, “variant” or “homologue” of a protein may optionally be characterised as having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of the reference protein (e.g. a reference isoform). In some embodiments fragments, variants, isoforms and homologues of a reference protein may be characterised by ability to perform a function performed by the reference protein.

A “fragment” generally refers to a fraction of the reference protein. A “variant” generally refers to a protein having an amino acid sequence comprising one or more (for example, 1, 2, 3, 4, 5, or more) amino acid substitutions, insertions, deletions or other modifications relative to the amino acid sequence of the reference protein, but retaining a considerable degree of sequence identity (e.g. at least 60%) to the amino acid sequence of the reference protein. An “isoform” generally refers to a variant of the reference protein expressed by the same species as the species of the reference protein. A “homologue” generally refers to a variant of the reference protein produced by a different species as compared to the species of the reference protein. For example, human B2M isoform 1 (P61769-1, v1; SEQ ID NO:1) and Rhesus macaque HERS (UniProt: Q6V7J5-1, v1; SEQ ID NO:4) are homologues of one another. Homologues include orthologues.

A “fragment” of a reference protein may be of any length (by number of amino acids), although may optionally be at least 20% of the length of the reference protein (that is, the protein from which the fragment is derived) and may have a maximum length of one of 50%, 75%, 80%, 85%, 90%, 91%, 92%, 93%, 94%, 95%, 96%, 97%, 98%, or 99% of the length of the reference protein. The amino acids in the minimum length may be contiguous with respect to any of the specific sequences disclosed herein.

A fragment of B2M may have a minimum length of one of 10, 20, 30, 40, 50, 75 or 100 amino acids, and may have a maximum length of one of 20, 30, 40, 50, 75 or 100 amino acids.

In some embodiments, the B2M is B2M from a mammal (e.g. a primate (rhesus, cynomolgous, non-human primate or human) and/or a rodent (e.g. rat or murine) B2M). Isoforms, fragments, variants or homologues of B2M may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature B2M isoform from a given species, e.g. human.

Isoforms, fragments, variants or homologues may optionally be functional isoforms, fragments, variants or homologues, e.g. having a functional property/activity of the reference B2M (e.g. human B2M), as determined by analysis by a suitable assay for the functional property/activity. For example, an isoform, fragment, variant or homologue of B2M may display association with an MHC class I α polypeptide.

In some embodiments the MHC class I α polypeptide association domain of the polypeptide of the present disclosure comprises, or consists of, or consists essentially of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of the Ig-like C1-type domain of B2M. In some embodiments the MHC class I α polypeptide association domain of the polypeptide of the present disclosure comprises, or consists of, or consists essentially of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of B2M.

In some embodiments the MHC class I α polypeptide association domain of the polypeptide of the present disclosure comprises, or consists of, or consists essentially of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:1, 2 or 3.

### Transmembrane Domain

The polypeptide of the present disclosure also comprises at least one transmembrane domain.

A transmembrane domain refers to any three-dimensional structure formed by a sequence of amino acids which is thermodynamically stable in a biological membrane, e.g. a cell membrane. The transmembrane domain may be an amino acid sequence which spans the cell membrane of a cell the polypeptide of the present disclosure.

The transmembrane domain may comprise or consist of or consist essentially of a sequence of amino acids which forms a hydrophobic alpha helix or beta-barrel. The amino acid sequence of the transmembrane domain of the polypeptide of the present disclosure may be, or may be derived from, the amino acid sequence of a transmembrane domain of a protein comprising a transmembrane domain.

Transmembrane domains are recorded in databases such as GenBank®, UniProt, Swiss-Prot, TrEMBL, Protein Information Resource, Protein Data Bank, Ensembl, and InterPro, and/or can be identified/predicted e.g. using amino acid sequence analysis tools such as TMHMM (Krogh et al., 2001 J Mol Biol 305: 567-580).

In some embodiments, the amino acid sequence of the transmembrane domain may be, or may be derived from, the amino acid sequence of the transmembrane domain of a protein expressed at the cell surface. In some embodiments the protein expressed at the cell surface is a receptor or ligand, e.g. an immune receptor or ligand. In some embodiments the amino acid sequence of the transmembrane domain may be, or may be derived from, the amino acid sequence of the transmembrane domain of one of ICOS, ICOSL, CD86, CTLA-4, CD28, CD80, MHC class I α, MHC class II α, MHC class II β, CD3c, CD3δ, CD3γ, CD3-ζ, TCRα TCRβ, CD4, CD8a, CD8β, CD40, CD40L, PD-1, PD-L1, PD-L2, 4-1 BB, 4-1BBL, OX40, OX40L, GITR, GITRL, TIM-3, Galectin 9, LAGS, CD27, CD70, LIGHT, HVEM, TIM-4, TIM-1, ICAM1, LFA-1, LFA-3, CD2, BTLA, CD160, LILRB4, LILRB2, VTCN1, CD2, CD48, 2B4, SLAM, CD30, CD30L, DR3, TL1A, CD226, CD155, CD112 and CD276. In some embodiments, the transmembrane domain is, or is derived from, the amino acid sequence of the transmembrane domain of CD8α, CD28, CD3-ζ, CD4, CD8β or CD226. In some embodiments, the transmembrane domain is not the transmembrane domain of CD3-ζ.

In some embodiments, the transmembrane domain of the polypeptide of the present disclosure comprises, or consists of, or consists essentially of, an amino acid sequence having at least 70%, 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:5. In some embodiments, the transmembrane domain of the polypeptide of the present disclosure comprises, or consists of, an amino acid sequence having at least 70%, 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:6.

### Signalling Domain

The polypeptide of the present disclosure comprises at least one signalling domain. The signalling domain provides sequences for initiating intracellular signalling in a cell expressing the polypeptide of the present disclosure.

The signalling domain comprises an immunoreceptor tyrosine-based activation motif (ITAM) containing sequence. ITAMs and ITAM-containing proteins are described e.g. in Love and Hayes, Cold Spring Harb Perspect Biol. (2010) 2(6): a002485, which is hereby incorporated by reference in its entirety. ITAMs comprise the amino acid sequence YXXL/I (SEQ ID NO:7), wherein “X” denotes any amino acid. In ITAM-containing proteins, sequences according to SEQ ID NO:7 are often separated by 6 to 8 amino acids; YXXL/I(X)₆₋₈YXXL/I (SEQ ID NO:8). When phosphate groups are added to the tyrosine residue of an ITAM by tyrosine kinases, a signalling cascade is initiated within the cell.

In some embodiments, the signalling domain of the polypeptide according to the present disclosure comprises one or more copies of an amino acid sequence according to SEQ ID NO:7 or SEQ ID NO:8. In some embodiments, the signalling domain comprises at least 1, 2, 3, 4, 5 or 6 copies of an amino acid sequence according to SEQ ID NO:7. In some embodiments, the signalling domain comprises at least 1, 2, or 3 copies of an amino acid sequence according to SEQ ID NO:8. In some embodiments, the signalling domain comprises 1 to 10, 2 to 8, 3 to 7 or 4 to 6 copies of an amino acid sequence according to SEQ ID NO:7. In some embodiments, the signalling domain comprises at least 1 to 6, 2 to 5, or 3 to 4 copies of an amino acid sequence according to SEQ ID NO:8.

In some embodiments, the signalling domain comprises an amino acid sequence which is, or which is derived from, the amino acid sequence of an ITAM-containing sequence of a protein having an ITAM-containing amino acid sequence. In some embodiments the signalling domain comprises an amino acid sequence which is, or which is derived from, an ITAM-containing sequence (e.g. the intracellular domain) of the amino acid sequence of one of CD3ε, CD3δ, CD3γ, CD79α, CD79β, FcγRI, FcγRIIA, FcγRIIC, FcγRIIIA, FcγRIV or DAP12. In some embodiments the signalling domain comprises an amino acid sequence which is, or which is derived from, an ITAM-containing sequence (e.g. the intracellular domain) of CD3-ζ. The intracellular domain of human CD3-ζ corresponds to positions 52-164 of the amino acid sequence of UniProt: P20963-1 (CD3Z_HUMAN), shown in SEQ ID NO:9.

In some embodiments, the signalling domain of the polypeptide of the present disclosure comprises, or consists of, or consists essentially of, an amino acid sequence having at least 70%, 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:9. In some embodiments, the signalling domain of the polypeptide of the present disclosure comprises an ITAM-containing amino acid sequence which comprises, or consists of, an amino acid sequence having at least 70%, 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:9.

In some embodiments the signalling domain of the polypeptide of the present disclosure comprises one or more costimulatory sequences derived from the signalling region of a costimulatory molecule. The one or more costimulatory sequences facilitate activation of the immune cell expressing the polypeptide upon ligation of the complex comprising MHC class I α and the polypeptide of the disclosure, by a T cell comprising a TCR capable of recognising the MHC class I complex. Costimulation promotes proliferation and survival of the cell expressing the polypeptide, and may also promote cytokine production, differentiation, cytotoxic function and memory formation. Molecular mechanisms of T cell costimulation are reviewed in Chen and Flies 2013 Nat Rev Immunol 13(4):227-242. Suitable co-stimulatory molecules include e.g. CD28, 4-1BB, OX40, ICOS and CD27.

In some embodiments, the costimulatory sequence is, or is derived from, the intracellular domain of a costimulatory molecule. In some embodiments, the costimulatory molecule is a member of the B7-CD28 superfamily (e.g. CD28, ICOS), or a member of the TNF receptor superfamily (e.g. 4-1 BB, OX40, CD27, DR3, GITR, CD30, HVEM). In some embodiments, the costimulatory sequence is, or is derived from, the intracellular domain of one of CD28, ICOS, 4-1 BB, CD27, OX40, HVEM, CD2, SLAM, TIM-1, CD30, GITR, DR3, LIGHT and CD226. In some embodiments, the signalling domain comprises a costimulatory sequence which is, or which is derived from, the intracellular domain of CD28.

Costimulatory molecules upregulate expression of genes promoting cell growth, effector function and survival through several transduction pathways. For example, CD28 and ICOS signal through phosphatidylinositol 3 kinase (PI3K) and AKT to upregulate expression of genes promoting cell growth, effector function and survival through NF-κB, mTOR, NFAT and AP1/2. CD28 also activates AP1/2 via CDCl₄2/RAC1 and ERK1/2 via RAS, and ICOS activates C-MAF. 4-1 BB, OX40, and CD27 recruit TNF receptor associated factor (TRAF) and signal through MAPK pathways, as well as through PI3K. Signalling domains of the polypeptide of the present disclosure may comprise costimulatory sequences derived from signalling regions of more than one co-stimulatory molecule.

In some embodiments, the signalling domain comprises a costimulatory sequence which comprises, or consists of, or consists essentially of, an amino acid sequence having at least 70%, 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:10.

### Additional Regions and Sequences

The polypeptide of the present disclosure may additionally comprise a spacer region between the MHC class I α polypeptide association domain and the transmembrane domain. A spacer region is an amino acid sequence which provides for flexible linkage of the MHC class I α polypeptide association domain and the transmembrane domain.

In some embodiments, the spacer region comprises, or consists of, an amino acid sequence which is, or which is derived from, the human IgG1 hinge region, the CH2CH3 (i.e. Fc) region of IgG1, the CH2 region of IgG1, the CH3 region of IgG1, IgG4, amino acids 187-189 of human IgD (Wilkie et al., 2008 J IMmunol 180(7): 4901-4909), a hinge region derived from CD8α, e.g. as described in WO 2012/031744 A1, or a hinge region derived from CD28, e.g. as described in WO 2011/041093 A1.

In some embodiments, the spacer region comprises, or consists of, or consists essentially of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:11.

In some embodiments, the polypeptide of the present disclosure may comprise a signal sequence (also known as a signal peptide or leader sequence). Signal sequences normally comprise a sequence of 5-30 hydrophobic amino acids, which form a single alpha helix. Secreted proteins and proteins expressed at the cell surface often comprise signal sequences. The signal sequence may be present at the N-terminus of the polypeptide, and may be present in the newly-synthesized polypeptide. The signal sequence provides for efficient trafficking of the polypeptide to the cell surface. Signal sequences are often removed by cleavage, and thus are not comprised in the mature polypeptide expressed at the cell surface.

Signal sequences are known for many proteins and are recorded in databases such as UniProt, GenBank®, Swiss-Prot, TrEMBL, Protein Information Resource, Protein Data Bank, Ensembl, and InterPro, and/or can be identified/predicted e.g. using amino acid sequence analysis tools such as SignalP (Petersen et al., 2011 Nature Methods 8: 785-786) or Signal-BLAST (Frank and Sippl, 2008 Bioinformatics 24: 2172-2176).

In some embodiments, the signal sequence of the polypeptide of the present disclosure comprises, or consists of, or consists essentially of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:12.

In some embodiments the polypeptides of the present disclosure additionally comprise one or more linker sequences between domains/regions of the polypeptide.

Linker sequences are known to the skilled person, and are described, for example in Chen et al., Adv Drug Deliv Rev (2013) 65(10): 1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen et al., Adv Drug Deliv Rev (2013) 65(10): 1357-1369. Flexible linker sequences often comprise high proportions of glycine and/or serine residues.

In some embodiments, a linker sequence according to the present disclosure comprises at least one glycine residue and/or at least one serine residue. In some embodiments the linker sequence consists of glycine and serine residues. In some embodiments, the linker sequence has a length of 1-2, 1-3, 1-4, 1-5 or 1-10 amino acids.

In some embodiments, the linker sequence may be a cleavable linker sequence. That is, the linker sequence may comprise a sequence of amino acids which is capable of being cleaved. For example, the linker sequence may comprise a sequence capable of acting as a substrate for an enzyme capable of cleaving peptide bonds—i.e. a cleavage site. Many such cleavage sites are known to and can be employed by the person skilled in the art of molecular biology. In some embodiments, the cleavable linker may comprise an autocleavage site. Autocleavage sites are automatically cleaved without the need for treatment with enzymes. An example of an autocleavage site is the 2A sequence from Picornavirus shown in SEQ ID NO:13, which is cleaved at “G/P”. SEQ ID NO:14 is an example of a larger linker sequence comprising the 2A autocleavage linker sequence.

In some embodiments the linker sequence comprises, or consists of, or consists essentially of, the amino acid sequence of SEQ ID NO:13. In some embodiments the detectable moiety comprises, or consists of, or consists essentially of, the amino acid sequence of SEQ ID NO:14, or an amino acid sequence having at least 70% amino acid sequence identity to SEQ ID NO:14.

The polypeptides of the present disclosure may additionally comprise further amino acids or sequences of amino acids. For example, the polypeptides may comprise amino acid sequence(s) to facilitate expression, folding, trafficking, processing, purification or detection of the polypeptide. For example, the antigen-binding molecule/polypeptide may comprise a sequence encoding a His, (e.g. 6XHis), Myc, GST, MBP, FLAG, HA, E, or Biotin tag, optionally at the N- or C-terminus of the polypeptide. In some embodiments the polypeptide comprises a detectable moiety, e.g. a fluorescent, lunminescent, immuno-detectable, radio, chemical, nucleic acid or enzymatic label, optionally at the N- or C-terminus of the polypeptide. Examples of detectable moieties that may be used in connection with the present disclosure are described e.g. in Philip et al., Blood (2014) 124:1277-1287, which is hereby incorporated by reference in entirety. For example, Philip et al., Blood (2014) 124:1277-1287 describes “Q8”, which is a detectable marker comprising a 16 amino acid epitope of CD34 fused to stalk region of CD8a.

In some embodiments the detectable moiety comprises, or consists of, the amino acid sequence of SEQ ID NO:15, or an amino acid sequence having at least 70% amino acid sequence identity to SEQ ID NO:15.

In some embodiments the polypeptide additionally comprises a peptide capable of forming a peptide:MHC class I molecule polypeptide complex. In some embodiments the polypeptide additionally comprises a peptide capable of being presented by an MHC class I α molecule. In some embodiments the peptide is connected via a linker (e.g. a flexible linker sequence or a cleavable linker sequence) to the N- or C-terminus of the polypeptide. In some embodiments the peptide is connected via a linker (e.g. a flexible linker sequence or a cleavable linker sequence) to the MHC class I α polypeptide association domain.

### Functional Properties of the Polypeptides of the Disclosure

Polypeptides of the present disclosure may be characterised by reference to certain functional properties.

In some embodiments the polypeptide according to the present disclosure may display association (e.g. may dimerise) with an MHC class I α polypeptide, e.g. at the surface (in or at the cell membrane) of a cell expressing the polypeptide and an MHC class I α polypeptide.

Assays for the functional properties described herein for the polypeptides and cells of the present disclosure may be performed in vitro, ex vivo or in vivo.

The ability of proteins to interact can be analysed by methods well known to the skilled person, such as fluorescence resonance energy transfer (FRET) and Bioluminescence Resonance Energy Transfer (BRET) assays using appropriate labelled interaction partners, e.g. as described in Ciruela, Curr Opin Biotechnol. (2008) 19(4):338-43. Association between the polypeptide of the present disclosure and an MHC class I α polypeptide may be non-covalent.

In some embodiments the polypeptide according to the present disclosure is capable of triggering/increasing one or more of the following by cell (e.g. an immune cell, e.g. a T cell, NK cell, or NKT cell) comprising/expressing the polypeptide, e.g. following exposure to a cell comprising/expressing a TCR specific for an MHC complex presented by the cell comprising/expressing the polypeptide: cell proliferation/population expansion, growth factor (e.g. IL-2) expression, cytotoxic/effector factor (e.g. IFNγ, granzyme, perforin, granulysin, CD107a, TNFα, FASL) expression and/or cytotoxic activity. In some embodiments a cell comprising/expressing the polypeptide may display reduced susceptibility to cell killing by a cell comprising/expressing a TCR specific for an MHC complex presented by the cell comprising/expressing the polypeptide.

Cell proliferation/population expansion can be investigated by analysing cell division or the number of cells over a period of time. Cell division can be analysed, for example, by in vitro analysis of incorporation of ³H-thymidine or by CFSE dilution assay, e.g. as described in Fulcher and Wong, Immunol Cell Biol (1999) 77(6): 559-564, hereby incorporated by reference in entirety. Proliferating cells can also be identified by analysis of incorporation of 5-ethynyl-2′-deoxyuridine (EdU) by an appropriate assay, as described e.g. in Buck et al., Biotechniques. 2008 June; 44(7):927-9, and Sali and Mitchison, PNAS USA 2008 Feb. 19; 105(7): 2415-2420, both hereby incorporated by reference in their entirety.

As used herein, “expression” may be gene or protein expression. Gene expression encompasses transcription of DNA to RNA, and can be measured by various means known to those skilled in the art, for example by measuring levels of mRNA by quantitative real-time PCR (qRT-PCR), or by reporter-based methods. Similarly, protein expression can be measured by various methods well known in the art, e.g. by antibody-based methods, for example by western blot, immunohistochemistry, immunocytochemistry, flow cytometry, ELISA, ELISPOT, or reporter-based methods.

Cytotoxicity and cell killing can be investigated, for example, using any of the methods reviewed in Zaritskaya et al., Expert Rev Vaccines (2011), 9(6):601-616, hereby incorporated by reference in its entirety. Examples of in vitro assays of cytotoxicity/cell killing assays include release assays such as the ⁵¹Cr release assay, the lactate dehydrogenase (LDH) release assay, the 3-(4,5-dimethylthiazol-2-yl)-2,5-diphenyl tetrazolium bromide (MTT) release assay, and the calcein-acetoxymethyl (calcein-AM) release assay. These assays measure cell killing based on the detection of factors released from lysed cells. Susceptibility to cell killing by a given cell type can be analysed e.g. by co-culturing the test cells with the given cell type, and measuring the number/proportion of cells viable/dead test cells after a suitable period of time.

In some embodiments the polypeptide according to the present disclosure is capable of increasing cell proliferation/population expansion, growth factor (e.g. IL-2) expression, cytotoxic/effector factor (e.g. IFNγ, granzyme, perforin, granulysin, CD107a, TNFα, FASL) expression and/or cytotoxic activity of a cell (e.g. an immune cell, e.g. a T cell, NK cell, or NKT cell) comprising/expressing the polypeptide following exposure to a cell comprising/expressing a TCR specific for an MHC complex presented by the cell comprising/expressing the polypeptide, as compared to the level displayed by a comparable cell not comprising/expressing the polypeptide. In some embodiments the polypeptide according to the present disclosure is capable of reducing susceptibility to cell killing by a cell comprising/expressing a TCR specific for an MHC complex presented by the cell comprising/expressing the polypeptide as compared to susceptibility to cell killing of a comparable cell not comprising/expressing the polypeptide.

A cell comprising/expressing a TCR specific for an MHC complex presented by the cell comprising/expressing the polypeptide may be present in a population of allogeneic cells, e.g. a population of cells obtained from a subject having an HLA profile which is different to the HLA profile of the cells comprising/expressing the polypeptide, nucleic acid/plurality of nucleic acids or an expression vector/plurality of expression vectors according to the present disclosure.

The increased cell proliferation/population expansion, growth factor (e.g. IL-2) expression, cytotoxic/effector factor (e.g. IFNγ, granzyme, perforin, granulysin, CD107a, TNFα, FASL) expression and/or cytotoxic activity may be one of greater than 1 times, e.g. ≥1.01 times, ≥1.02 times, ≥1.03 times, 1.04 times, ≥1.05 times, ≥1.06 times, ≥1.07 times, ≥1.08 times, ≥1.09 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥2.1 times, ≥2.2 times, ≥2.3 times, ≥2.4 times, ≥2.5 times, ≥2.6 times, ≥2.7 times, ≥2.8 times, ≥2.9 times, ≥3 times, ≥3.5 times, 4 times, ≥4.5 times, ≥5 times, ≥6 times, ≥7 times, ≥8 times, ≥9 times, ≥10 times, ≥15 times, ≥20 times, ≥25 times, ≥30 times, ≥35 times, ≥40 times, ≥45 times, ≥50 times, ≥60 times, ≥70 times, ≥80 times, ≥90 times, ≥100 times, ≥200 times, ≥300 times, ≥400 times, ≥500 times, ≥600 times, ≥700 times, ≥800 times, ≥900 times, ≥1000 times the level of cell proliferation/population expansion, growth factor (e.g. IL-2) expression, cytotoxic/effector factor (e.g. IFNγ, granzyme, perforin, granulysin, CD107a, TNFα, FASL) expression, cytotoxic activity displayed by a comparable cell not comprising/expressing the polypeptide, under the same conditions.

Reduced susceptibility to cell killing may be determined by detection of a level of cell killing which is less than 1 times, e.g. one of ≤0.99 times, ≤0.95 times, ≤0.9 times, ≤0.85 times, ≤0.8 times, ≤0.85 times, ≤0.75 times, ≤0.7 times, ≤0.65 times, ≤0.6 times, ≤0.55 times, ≤0.5 times, ≤0.45 times, ≤0.4 times, ≤0.35 times, 0.3 times, ≤0.25 times, ≤0.2 times, ≤0.15 times, ≤0.1 times, ≤0.09 times, ≤0.08 times, ≤0.07 times, ≤0.06 times, ≤0.05 times, ≤0.04 times, ≤0.03 times, ≤0.02 times or ≤0.01 times the level of cell killing of comparable cells not comprising/expressing the polypeptide, under the same conditions.

### Nucleic Acids and Vectors of the Disclosure

The present disclosure also provides nucleic acids encoding a polypeptide according to the present disclosure. Any polynucleotides of the present disclosure may be non-natural, including synthetic. They may be recombinantly generated.

In some embodiments, the nucleic acid is purified or isolated, e.g. from other nucleic acid, or naturally-occurring biological material. In some embodiments the nucleic acid(s) comprise or consist of DNA and/or RNA. The present disclosure also provides a vector comprising the nucleic acid according to the present disclosure.

The nucleotide sequence may be contained in a vector, e.g. an expression vector. A “vector” as used herein is a nucleic acid molecule used as a vehicle to transfer exogenous nucleic acid into a cell. The vector may be a vector for expression of the nucleic acid in the cell. Such vectors may include a promoter sequence operably linked to the nucleotide sequence encoding the sequence to be expressed. A vector may also include a termination codon and expression enhancers. Any suitable vectors, promoters, enhancers and termination codons known in the art may be used to express a peptide or polypeptide from a vector according to the disclosure.

Suitable vectors include plasmids, binary vectors, DNA vectors, mRNA vectors, viral vectors (e.g. retroviral vectors, gammaretroviral vectors (e.g. murine Leukemia virus (MLV)-derived vectors), lentiviral vectors, adenovirus vectors, adeno-associated virus vectors, vaccinia virus vectors and herpesvirus vectors), transposon-based vectors, and artificial chromosomes (e.g. yeast artificial chromosomes), e.g. as described in Maus et al., Annu Rev Immunol (2014) 32:189-225 or Morgan and Boyerinas, Biomedicines 2016 4, 9, which are both hereby incorporated by reference in its entirety.

In this specification the term “operably linked” may include the situation where a selected nucleic acid sequence and regulatory nucleic acid sequence (e.g. promoter and/or enhancer) are covalently linked in such a way as to place the expression of the nucleotide sequence under the influence or control of the regulatory sequence (thereby forming an expression cassette). Thus a regulatory sequence is operably linked to the selected nucleic acid sequence if the regulatory sequence is capable of effecting transcription of the nucleic acid sequence. Where appropriate, the resulting transcript may then be translated into a desired polypeptide.

In some embodiments the nucleic acid/vector encoding a polypeptide of the present disclosure may comprise one or more sequences for controlling expression of the polypeptide. A sequence for controlling expression of the polypeptide may provide for expression of the polypeptide by cells comprising the nucleic acid/vector in response to e.g. an agent/signal, with the result that expression of the polypeptide can be controlled. The sequence for controlling expression of the polypeptide may be tissue-specific, in some embodiments.

In some embodiments the nucleic acid/vector encoding a polypeptide of the present disclosure comprises at least one control element for inducible upregulation of expression of the polypeptide. For example, the nucleic acid may comprise a control element for inducible upregulation of expression of the polypeptide from the nucleic acid/expression vector in response to treatment with a particular agent. The agent may provide for inducible upregulation of expression of the polypeptide in vivo by administration of the agent to a subject having been administered with a modified cell according to the disclosure, or ex vivo/in vitro by administration of the agent to cells in culture ex vivo or in vitro.

In some embodiments the nucleic acid(s)/vector(s) employ a conditional expression system for controlling expression of the polypeptide by cells comprising the nucleic acid(s)/vector(s). The inventors demonstrate herein that cells comprising nucleic acid(s)/vector(s) employing a conditional expression system for controlling expression of the polypeptide proliferate/expand with greater efficiency as compared to cells constitutively expressing the polypeptide of the disclosure. “Conditional expression” may also be referred to herein as “inducible expression”, and refers to gene/protein expression contingent on certain conditions, e.g. the presence of a particular agent.

Accordingly, in some embodiments the nucleic acid/vector comprises nucleic acid sequence encoding a conditional expression system for controlling expression of the polypeptide by cells comprising the nucleic acid/vector. Conditional expression systems are well known in the art and are reviewed e.g. in Ryding et al. Journal of Endocrinology (2001) 171, 1-14, which is hereby incorporated by reference in its entirety.

Conditional expression systems include systems which employ tetracycline-controlled transcriptional activation, such as Tet-On and Tet-Off systems.

The Tet-On system employs nucleic acid encoding a reverse tetracycline transactivator (rtTA) protein, which is a fusion of the tetracycline repressor (TetR) protein mutated at four amino acid positions to reverse the response to tetracycline/doxycycline, and the activation domain of VP16. In the absence of tetracycline (or a derivative thereof such as doxycycline) rtTA does not bind to TetO operator sequences and the polypeptide is not expressed. In the presence of tetracycline/doxycycline, rtTA binds to TetO sequences in the TRE and activates transcription of the nucleic acid downstream of the promoter. Tet-On systems are described in Das et al., Curr Gene Ther. (2016)16(3):156-67 (hereby incorporated by reference in its entirety), and include systems using optimised rtTA variants such as the Tet-On Advanced system (which uses the rtTA variant protein rtTA2^{s}-M2) and Tet-On 3G system.

The Tet-On Advanced system is also described in Urlinger et al. Proc. Natl. Acad. Sci. U.S.A. (2000) 97(14):7963-8 (hereby incorporated by reference in entirety), and Tet-On 3G is described in Zhou et al., Gene Ther. 13(19):1382-1390 (hereby incorporated by reference in entirety).

The Tet-Off system employs nucleic acid encoding a tetracycline transactivator (tTA) protein, which is a fusion of the tetracycline repressor (TetR) protein and the activation domain HSV protein VP16. In the absence of tetracycline (or a derivative thereof such as doxycycline) tTA binds to TetO operator sequences, which form a tetracycline-response element (TRE), located just upstream of a minimal promoter (e.g. CMV promoter). Binding of tTA to the TetO sequences in the TRE activates transcription of the nucleic acid downstream of the promoter. In the presence of tetracycline/doxycycline tTA is unable to bind TetO sequences in the TRE, and transcription of the nucleic acid downstream of the promoter is repressed. The Tet-Off system is described in Bujard et al. Proc. Natl. Acad. Sci. U.S.A. (1992) 89(12):5547-51 (hereby incorporated by reference in entirety).

Other tetracycline-controlled systems include the T-REx conditional expression system described in Yao et al., Human Gene Therapy (1998) 9(13): 1939-1950 (hereby incorporated by reference in entirety). In the T-REx system, TetR is expressed under the control of a CMV promoter, and in the absence of tetracycline/doxycycline TetR binds to two Tet operator 2 (TetO2) sequences upstream of the region of interest and repression of transcription of the region of interest. When tetracycline/doxycycline is added to the system it binds to TetR and causes its release from the TetO2 sequences, thereby releasing the region of interest from transcriptional repression.

In some embodiments the nucleic acids/vectors of the present disclosure comprise nucleic acid sequence encoding elements of a system for providing conditional expression of the polypeptide of the present disclosure. In some embodiments the nucleic acid/vector encodes a tetracycline-controlled transcriptional activation system for controlling expression of the polypeptide. In some embodiments the nucleic acid/vector comprises sequence encodes a Tet-On system (e.g. Tet-On Advanced system or Tet-On 3G system) for controlling expression of the polypeptide.

In some embodiments the nucleic acids/vectors of the present disclosure comprise nucleic acid sequence encoding a peptide/peptides capable of forming a peptide:MHC class I molecule polypeptide complex. In some embodiments the nucleic acids/vectors comprise nucleic acid sequence encoding a peptide/peptides capable of being presented by an MHC class I α molecule.

### Cells Comprising the Polypeptides/Nucleic Acids/Vectors of the Disclosure

The present disclosure also provides a cell comprising/expressing a polypeptide according to the present disclosure. Also provided is a cell comprising/expressing a nucleic acid/plurality of nucleic acids or an expression vector/plurality of expression vectors according to the present disclosure. It will be appreciated that “a cell” as used herein encompasses plural cells, e.g. populations of such cells.

The cell comprising/expressing a polypeptide according to the present disclosure or comprising a nucleic acid/plurality of nucleic acids or an expression vector/plurality of expression vectors according to the present disclosure may be a eukaryotic cell, e.g. a mammalian cell. The mammal may be a human, or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate). The cell may be a prokaryotic cell, for example E. coli, such as in cases wherein the polynucleotide(s) or polypeptide(s) encompassed by the present disclosure are produced and/or stored and/or sold.

In some embodiments, the cell may be from, or may have been obtained from, a human subject. Where the cell is to be administered to a subject, or where the cell is to be used in the preparation of a population of cells to be administered to a subject, the cell may be from a different subject to the subject to be administered (i.e. the cell may be allogeneic).

In some embodiments the cell has an HLA type which is different to the HLA type of the subject to be administered. In some embodiments the cell has an HLA type which is the same as the HLA type of the subject to be administered.

The cell may be an immune cell. The cell may be a cell of hematopoietic origin, e.g. a neutrophil, eosinophil, basophil, dendritic cell, lymphocyte, or monocyte. The lymphocyte may be e.g. a T cell, B cell, NK cell, NKT cell or innate lymphoid cell (ILC), or a precursor thereof. The cell may express e.g. CD3 polypeptides (e.g. CD3γ CD3ε CD3ζ or CD3δ), TCR polypeptides (TCRα or TCRβ), CD27, CD28, CD4 and/or CD8.

In some embodiments, the cell is a T cell. In some embodiments, the T cell is a CD3+ T cell. In some embodiments, the T cell is a CD3+, CD4+ T cell. In some embodiments, the T cell is a CD3+, CD8+ T cell. In some embodiments, the T cell is a T helper cell (TH cell)). In some embodiments, the T cell is a cytotoxic T cell (e.g. a cytotoxic T lymphocyte (CTL)).

In some embodiments the cell comprising/expressing a polypeptide, nucleic acid/plurality of nucleic acids or an expression vector/plurality of expression vectors according to the present disclosure is specific for an antigen of interest. In some embodiments the cell comprises/expresses an antigen receptor specific for an antigen of interest. In some embodiments the antigen receptor is a T cell receptor (TCR). In some embodiments the antigen receptor is a chimeric antigen receptor (CAR). CARs are recombinant receptors that provide both antigen-binding and T cell activating functions. CAR structure and engineering is reviewed, for example, in Dotti et al., Immunol Rev (2014) 257(1), hereby incorporated by reference in its entirety. CARs comprise an antigen-binding region linked to a cell membrane anchor region and a signalling region. An optional hinge region may provide separation between the antigen-binding region and cell membrane anchor region, and may act as a flexible linker.

In some embodiments, the antigen is a cancer cell antigen, including a tumor antigen. In some embodiments the antigen is or is part of a receptor molecule, e.g. a cell surface receptor. In some embodiments the antigen is a cell signalling molecule, e.g. a cytokine, chemokine, interferon, interleukin or lymphokine. In some embodiments the antigen is a growth factor or a hormone. In some embodiments the antigen is a molecule expressed by an allogeneic cell/tissue/organ (e.g. a cell/tissue/organ having an HLA profile which is different to the HLA profile of a cell comprising/expressing the polypeptide, nucleic acid/plurality of nucleic acids or an expression vector/plurality of expression vectors according to the present disclosure).

A cancer cell antigen is an antigen which is expressed or over-expressed by a cancer cell. A cancer cell antigen may be any peptide/polypeptide, glycoprotein, lipoprotein, glycan, glycolipid, lipid, or fragment thereof. A cancer cell antigen's expression may be associated with a cancer. A cancer cell antigen may be abnormally expressed by a cancer cell (e.g. the cancer cell antigen may be expressed with abnormal localisation), or may be expressed with an abnormal structure by a cancer cell. A cancer cell antigen may be capable of eliciting an immune response. In some embodiments, the antigen is expressed at the cell surface of the cancer cell (i.e. the cancer cell antigen is a cancer cell surface antigen). In some embodiments, the part of the antigen which is bound by the antigen-binding molecule described herein is displayed on the external surface of the cancer cell (i.e. is extracellular). The cancer cell antigen may be a cancer-associated antigen. In some embodiments the cancer cell antigen is an antigen whose expression is associated with the development, progression or severity of symptoms of a cancer. The cancer-associated antigen may be associated with the cause or pathology of the cancer, or may be expressed abnormally as a consequence of the cancer. In some embodiments, the cancer cell antigen is an antigen whose expression is upregulated (e.g. at the RNA and/or protein level) by cells of a cancer, e.g. as compared to the level of expression of by comparable non-cancerous cells (e.g. non-cancerous cells derived from the same tissue/cell type). In some embodiments, the cancer-associated antigen may be preferentially expressed by cancerous cells, and not expressed by comparable non-cancerous cells (e.g. non-cancerous cells derived from the same tissue/cell type). In some embodiments, the cancer-associated antigen may be the product of a mutated oncogene or mutated tumor suppressor gene. In some embodiments, the cancer-associated antigen may be the product of an overexpressed cellular protein, a cancer antigen produced by an oncogenic virus, an oncofetal antigen, or a cell surface glycolipid or glycoprotein.

In some embodiments, the cell comprising/expressing a polypeptide, nucleic acid/plurality of nucleic acids or an expression vector/plurality of expression vectors according to the present disclosure is an antigen-specific T cell. In embodiments herein, an “antigen-specific” T cell is a cell which displays certain functional properties of a T cell in response to the antigen for which the T cell is specific, or a cell expressing said antigen. The T cell may comprise a TCR specific for the antigen, or may comprise a CAR specific for the antigen. In some embodiments, the properties are functional properties associated with effector T cells, e.g. cytotoxic T cells.

In some embodiments, an antigen-specific T cell may display one or more of the following properties (e.g. in response to contact with a cell comprising/expressing antigen for which the T cell is specific): cytotoxicity, proliferation, IFNγ expression, CD107a expression, IL-2 expression, TNFα expression, perforin expression, granzyme expression, granulysin expression, and/or FAS ligand (FASL) expression. Antigen-specific T cells comprise a TCR capable of recognising a peptide of the antigen for which the T cell is specific when presented by the appropriate MHC molecule. Antigen-specific T cells may be CD4+ T cells and/or CD8+ T cells.

In some embodiments, the antigen for which the T cell is specific may be a peptide or polypeptide of a virus. T cells which are specific for an antigen of a virus may be referred to herein as a virus-specific T cell (VST). VSTs may be CD4+ T cells (e.g. TH cells) and/or CD8+ T cells (e.g. CTLs). A T cell which is specific for an antigen of a particular virus may be described as being specific for the relevant virus; for example, a T cell which is specific for an antigen of Epstein-Barr virus (EBV) may be referred to as an EBV-specific T cell, or “EBVST”.

An advantage associated with the use of virus-specific T cells for the generation of cells expressing the polypeptide of the present disclosure is that whilst naïve T cells may have limited long-term persistence after infusion, virus-specific T cells (VSTs) are derived from the memory compartment, and genetically-modified VSTs have been shown to persist for over 10 years after infusion in stem cell transplant recipients (Cruz et al., Cytotherapy (2010) 12:743-749). For example, VSTs expressing GD2.CARs have been shown to persist long-term after infusion and produce complete tumor responses in patients with low tumor burden (Sun et al., Journal for Immunotherapy of Cancer (2015) 3:5 and Pule et al., Nature Medicine (2008) 14: 1264-1270).

The present inventors have determined that virus-specific T cells comprising/expressing a polypeptide according to the present disclosure or comprising a nucleic acid/plurality of nucleic acids or an expression vector/plurality of expression vectors according to the present disclosure are particularly effective at eliminating alloreactive T cells.

In some embodiments the cell comprising/expressing a polypeptide according to the present disclosure or comprising a nucleic acid/plurality of nucleic acids or an expression vector/plurality of expression vectors according to the present disclosure is a virus-specific T cell (VST, e.g. a virus-specific CD4+ T cell (e.g. TH cell) and/or a virus-specific CD8+ T cell (e.g. CTL). In some embodiments the virus for which the T cell is specific is selected from Epstein-Barr virus (EBV), human papilloma virus (HPV), Adenovirus, Cytomegalovius (CMV), influenza virus, measles virus, hepatitis B virus (HBV), hepatitis C virus (HCV), human immunodeficiency virus (HIV), lymphocytic choriomeningitis virus (LCMV), or herpes simplex virus (HSV).

In some embodiments the cell comprising/expressing a polypeptide, nucleic acid/plurality of nucleic acids or an expression vector/plurality of expression vectors according to the present disclosure is an Epstein-Barr virus-specific T cell (EBVST), human papilloma virus-specific T cell (HPVST), Adenovirus-specific T cell (AdVST), Cytomegalovius-specific T cell (CMVST), influenza virus-specific T cell, measles virus-specific T cell, hepatitis B virus-specific T cell (HBVST), hepatitis C virus-specific T cell (HCVST), human immunodeficiency virus-specific T cell (HIVST), lymphocytic choriomeningitis virus-specific T cell (LCMVST), or Herpes simplex virus-specific T cell (HSVST).

In some embodiments the cell comprising/expressing a polypeptide, a nucleic acid/plurality of nucleic acids or an expression vector/plurality of expression vectors according to the present disclosure comprises modification to increase expression/activity of one or more factors capable of inhibiting apoptosis (i.e. of the cell modified to increase expression/activity of the relevant factor). Cells so modified are demonstrated herein to have improved survival/persistence, and to be less susceptible to apoptosis (e.g. apoptosis mediated by effector immune cells).

Mechanisms of apoptosis are reviewed e.g. in Elmore, Toxicol Pathol. (2007) 35(4):495-516, which is hereby incorporated by reference in its entirety. The three pathways for apoptosis are the intrinsic pathway, the extrinsic pathway and the perforin/granzyme pathway. The intrinsic pathway is activated by stress such as DNA damage, toxicity, hypoxia, protein misfolding etc. Under such conditions leakage of cytochrome c from cellular mitochondria into the cytoplasm, leading to caspase activation. The extrinsic pathway is triggered when a cell receives death signals from another cell, by ligation of death receptors such as Fas and TNFR expressed at the cell surface. Signalling through the death receptors leads to caspase activation. The perforin/granzyme pathway is activated by performs and granzymes produced e.g. by cytotoxic T cells. Perforins create holes in the cell, and granzymes enter the cell and activate caspases.

In some embodiments the cell comprises nucleic acid (e.g. exogenous nucleic acid) encoding one or more factors capable of inhibiting apoptosis. Factors capable of inhibiting apoptosis include e.g. cFLIP, PI-9, IAP proteins (e.g. cIAP1, cIAP2, XIAP, BIRC7, NAIP, survivin) and BCL2. In some embodiments the cell comprises nucleic acid (e.g. exogenous nucleic acid) encoding cFLIP or a variant thereof and/or PI-9 or a variant thereof.

SEQ ID NOs:24 and 25 respectively show the amino acid sequences of variants of cFLIP and PI-9, engineered for increased protein stability. SEQ ID NO:24 comprises the substitution K167R relative to UniProt:O15519-1. SEQ ID NO:25 comprises the substitutions C341S and C342S relative to UniProt:P50453-1.

In some embodiments the cell comprises nucleic acid (e.g. exogenous nucleic acid) encoding an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:24.

In some embodiments the cell comprises nucleic acid (e.g. exogenous nucleic acid) encoding an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:25.

In some embodiments the cell comprises nucleic acid (e.g. exogenous nucleic acid) having at least 80%, 85% 86%, 87%, 88%, 89%, 90%, 91%, 92%, 93%, 94%, 95%, 96%, 97%, 98%, 99%, or 100% sequence identity to the sequence of SEQ ID NO:26 or an equivalent sequence as a result of codon degeneracy.

In some embodiments the cell comprises nucleic acid (e.g. exogenous nucleic acid) having at least 80%, 85% 86%, 87%, 88%, 89%, 90%, 91%, 92%, 93%, 94%, 95%, 96%, 97%, 98%, 99%, or 100% sequence identity to the sequence of SEQ ID NO:27 or an equivalent sequence as a result of codon degeneracy.

Factors capable of inhibiting apoptosis include factors capable of reducing the expression and/or activity of one or more components/effectors of an apoptotic pathway (e.g. an inhibitor of, or antisense nucleic acid for, one or more components/effectors of an apoptosis pathway). Components/effectors of apoptosis pathways include death receptors (e.g. Fas, TNFR1, TNFR2, DR3, DR4, DR5, DR6), ligands for death receptors (e.g. FasL, TNFα, TRAIL), adaptor and signal-transduction proteins involved in apoptosis (e.g. TRAF proteins, TRADD, FADD, RIP1K), granzymes (e.g. granzyme B) and caspases (e.g. caspases 3, 6, 7, 8, 9, 10 etc.).

In some embodiments the cell comprising/expressing a polypeptide, a nucleic acid/plurality of nucleic acids or an expression vector/plurality of expression vectors according to the present disclosure comprises modification to increase expression/activity of one or more factors capable of inhibiting apoptosis triggered via the extrinsic and/or perforin/granzyme pathway.

### Methods for Producing Cells Comprising/Expressing the Polypeptides/Nucleic Acids/Vectors of the Disclosure

Aspects of the present disclosure comprise modifying an immune cell to express or comprise a polypeptide, a nucleic acid/plurality of nucleic acids or an expression vector/plurality of expression vectors according to the present disclosure.

In some embodiments the methods for producing cells comprising/expressing a polypeptide, a nucleic acid/plurality of nucleic acids or an expression vector/plurality of expression vectors according to the present disclosure comprise introducing a nucleic acid/plurality of nucleic acids or an expression vector/plurality of expression vectors according to the present disclosure into a cell. In some embodiments the cell is a cell as described herein. In some embodiments introducing a nucleic acid/plurality of nucleic acids or an expression vector/plurality of expression vectors into a cell comprises transfection or transduction, e.g. retroviral transduction. Accordingly, in some embodiments the nucleic acid/plurality is comprised in a viral vector, or the expression vector is a viral vector. In some embodiments, the method comprises introducing a nucleic acid or vector according into a cell by electroporation, e.g. as described in Koh et al., Molecular Therapy—Nucleic Acids (2013) 2, e114, which is hereby incorporated by reference in its entirety.

Aspects of the present disclosure comprise culturing a cell comprising a nucleic acid or a plurality of nucleic acids, or an expression vector or a plurality of expression vectors according to the present disclosure, under conditions suitable for expression of the polypeptide from the nucleic acid(s) or expression vector(s).

Culture of cells in accordance with the methods of the disclosure is performed using suitable medium and under suitable environmental conditions (e.g. temperature, pH, humidity, atmospheric conditions, agitation etc.) for the in vitro culture of immune cells, which are well known to the person skilled in the art of cell culture.

Conveniently, cultures of cells may be maintained at 37° C. in a humidified atmosphere containing 5% CO₂. Cultures can be performed in any vessel suitable for the volume of the culture, e.g. in wells of a cell culture plate, cell culture flasks, a bioreactor, etc. The cell cultures can be established and/or maintained at any suitable density, as can readily be determined by the skilled person. For example, cultures may be established at an initial density of ˜0.5×10⁶ to ˜5×10⁶ cells/ml of the culture (e.g. ˜1×10⁶ cells/ml). Cells may be cultured in any suitable cell culture vessel. In some embodiments of the methods according to the various aspects of the present disclosure, cells are cultured in a bioreactor. In some embodiments, cells are cultured in a bioreactor described in Somerville and Dudley, Oncoimmunology (2012) 1(8):1435-1437, which is hereby incorporated by reference in its entirety. In some embodiments cells are cultured in a GRex cell culture vessel, e.g. a GRex flask or a GRex 100 bioreactor.

Immune cells used in the methods may be derived e.g. from a blood sample, peripheral blood mononuclear cells (PBMCs), peripheral blood lymphocytes (PBLs) or tumor infiltrating lymphocytes (TILs). The immune cells used in the methods of the disclosure may be freshly isolated from a subject (or a sample isolated therefrom), or may be thawed from a sample which has previously been obtained and frozen.

In some embodiments, the methods for producing (e.g. generating or expanding) cells comprising/expressing a polypeptide, a nucleic acid/plurality of nucleic acids or an expression vector/plurality of expression vectors according to the present disclosure comprise:
(a) isolating immune cells from a subject;
(b) modifying at least one immune cell to express or comprise a polypeptide, a nucleic acid or plurality of nucleic acids, or an expression vector or plurality of expression vectors according to the present disclosure; and
(c) optionally expanding the modified at least one immune cell.

In some embodiments the method steps for production of cells comprising/expressing a polypeptide, a nucleic acid/plurality of nucleic acids or an expression vector/plurality of expression vectors according to the present disclosure may comprise one or more of: isolating immune cells from a subject; taking a blood sample from a subject; isolating PBMCs from a blood sample; modifying at least one immune cell to express or comprise a polypeptide, a nucleic acid or plurality of nucleic acids, or an expression vector or plurality of expression vectors, e.g. by introducing a nucleic acid/plurality of nucleic acids or an expression vector/plurality of expression vectors according to the present disclosure into at least one immune cell; culturing the modified at least one immune cell in in vitro or ex vivo cell culture; generating or expanding a population of modified immune cells; inducing expression of the polypeptide e.g. using a control element and/or a conditional expression system; collecting the modified immune cells.

In some embodiments the immune cells modified to express or comprise a polypeptide, a nucleic acid or plurality of nucleic acids, or an expression vector or plurality of expression vectors according to the present disclosure are immune cells specific for antigen(s) of interest and/or a virus of interest. Methods for generating/expanding populations of immune cells specific for antigen(s) of interest and/or a virus of interest are well known in the art, and are described e.g. in Wang and Riviére Cancer Gene Ther. (2015) 22(2):85-94, which is hereby incorporated by reference in its entirety. Such methods may involve contacting heterogeneous populations of immune cells (e.g. peripheral blood mononuclear cells (PBMCs), peripheral blood lymphocytes (PBLs), tumor-infiltrating lymphocytes (TILs)) with one or more peptides of the antigen(s) of interest, or cells comprising/expressing the antigen(s)/peptides. Cells comprising/expressing the antigen(s)/peptides may do so as a consequence of infection with the virus comprising/encoding the antigen(s), uptake by the cell of the antigen(s)/peptides thereof or expression of the antigen(s)/peptides thereof. The presentation is typically in the context of an MHC molecule at the cell surface of the antigen-presenting cell.

Cells comprising/expressing the antigen(s)/peptides may have been contacted (“pulsed”) with peptides of the antigen(s) according to methods well known to the skilled person. Antigenic peptides may be provided in a library of peptide mixtures (corresponding to one or more antigens), which may be referred to as pepmixes. Peptides of pepmixes may e.g. be overlapping peptides of 8-20 amino acids in length, and may cover all or part of the amino acid sequence of the relevant antigen. In some embodiments the overlapping peptides have overlapping regions of at least 2, 3, 4, 5, 6, 7, 8, 9, 10, 11, 12, 13, 14, 15, 16, 17, 18, or 19 amino acids in length, or a mixture thereof.

Cells within the population of immune cells comprising receptors specific for the peptide(s) may be activated (and stimulated to proliferate), following recognition of peptide(s) of the antigen(s) presented by antigen-presenting cells (APCs) in the context of appropriate costimulatory signals. It will be appreciated that “an immune cell specific for a virus” encompasses plural cells, e.g. populations of such cells. Such populations may be generated/expanded in vitro and/or ex vivo.

In some embodiments an immune cell specific for a virus may be generated/expanded (or may have been generated/expanded) by a method comprising: stimulating a population of immune cells by culture in the presence of antigen presenting cells (APCs) presenting a peptide of the virus. In some embodiments the immune cell specific for a virus may be modified to express or comprise a polypeptide, a nucleic acid/plurality of nucleic acids or an expression vector/plurality of expression vectors according to the present disclosure.

In some embodiments, provided is a method of producing (e.g. generating or expanding) a population of virus-specific immune cells, comprising:
(a) isolating immune cells from a subject;
(b) generating or expanding a population of virus-specific immune cells by a method comprising: stimulating the immune cells by culture in the presence of antigen presenting cells (APCs) presenting a peptide of the virus;
(c) modifying at least one virus-specific immune cell to express or comprise a polypeptide, a nucleic acid or plurality of nucleic acids, or an expression vector or plurality of expression vectors according to the present disclosure; and
(d) optionally expanding the modified at least one virus-specific immune cell.

In some embodiments the method steps for production of an immune cell specific for a virus may comprise one or more of: isolating immune cells from a subject; taking a blood sample from a subject; isolating PBMCs from a blood sample; generating/expanding a population of immune cells specific for a virus (e.g. by culturing PBMCs in the presence of cells (e.g. APCs) comprising/expressing antigen(s)/peptide(s) of the virus); culturing immune cells specific for a virus in in vitro or ex vivo cell culture; collecting immune cells specific for a virus.

In some embodiments the methods for producing a cell according to the present disclosure further comprise modifying the cell to increase expression/activity of one or more factors capable of inhibiting apoptosis. In some embodiments the methods comprise introducing nucleic acid (e.g. exogenous nucleic acid) encoding one or more factors capable of inhibiting apoptosis into the cell. In some embodiments the methods comprise introducing one or more factors capable of reducing the expression and/or activity of one or more components/effectors of an apoptotic pathway into the cell. In some embodiments the methods comprise introducing into a cell nucleic acid (e.g. exogenous nucleic acid) encoding cFLIP and/or PI-9.

Also provided are methods comprising culturing a cell according to the present disclosure, e.g. in vitro/ex vivo, e.g. for generating/expanding a population of such cells. Also provided are cells and populations of cells obtained or obtainable by the methods for producing a cell according to the present disclosure.

### Functional Properties of Cells Comprising/Expressing the Polypeptides/Nucleic Acids/Vectors of the Disclosure

Cells comprising/expressing a polypeptide, a nucleic acid/plurality of nucleic acids or an expression vector/plurality of expression vectors according to the present disclosure may be characterised by reference to one or more functional properties.

In some embodiments cells (e.g. immune cells, e.g. T cells, NK cells) comprising/expressing a polypeptide, a nucleic acid/plurality of nucleic acids or an expression vector/plurality of expression vectors according to the present disclosure display one or more of the following properties following exposure to a cell comprising/expressing a TCR specific for an MHC complex presented by the cell: cell proliferation/population expansion, growth factor (e.g. IL-2) expression, cytotoxic/effector factor (e.g. IFNγ, granzyme, perforin, granulysin, CD107a, TNFα, FASL) expression and/or cytotoxic activity. In some embodiments, cells comprising/expressing a polypeptide, a nucleic acid/plurality of nucleic acids or an expression vector/plurality of expression vectors according to the present disclosure may display reduced susceptibility to cell killing by a cell comprising/expressing a TCR specific for an MHC complex presented by the cell.

A cell comprising/expressing a TCR specific for an MHC complex presented by the cells may be present in a population of allogeneic cells, e.g. a population of cells obtained from a subject having an HLA profile which is different to the HLA profile of the cells comprising/expressing the polypeptide, nucleic acid/plurality of nucleic acids or an expression vector/plurality of expression vectors according to the present disclosure.

In some embodiments the cells comprising/expressing a polypeptide, a nucleic acid/plurality of nucleic acids or an expression vector/plurality of expression vectors according to the present disclosure display increased cell proliferation/population expansion, growth factor (e.g. IL-2) expression, cytotoxic/effector factor (e.g. IFNγ, granzyme, perforin, granulysin, CD107a, TNFα, FASL) expression and/or cytotoxic activity following exposure to a cell comprising/expressing a TCR specific for an MHC complex presented by the cell, as compared to the level of cell proliferation/population expansion, growth factor (e.g. IL-2) expression, cytotoxic/effector factor (e.g. IFNγ, granzyme, perforin, granulysin, CD107a, TNFα, FASL) expression, cytotoxic activity of a comparable cell not comprising/expressing a polypeptide, a nucleic acid/plurality of nucleic acids or an expression vector/plurality of expression vectors according to the present disclosure. In some embodiments the cells comprising/expressing a polypeptide, a nucleic acid/plurality of nucleic acids or an expression vector/plurality of expression vectors according to the present disclosure display reduced susceptibility to cell killing by a cell comprising/expressing a TCR specific for an MHC complex presented by the cell, as compared to susceptibility to cell killing displayed by comparable cells not comprising/expressing the polypeptide, nucleic acid/plurality of nucleic acids or expression vector/plurality of expression vectors according to the present disclosure.

The increased cell proliferation/population expansion, growth factor (e.g. IL-2) expression, cytotoxic/effector factor (e.g. IFNγ, granzyme, perforin, granulysin, CD107a, TNFα, FASL) expression or cytotoxic activity may be one of greater than 1 times, e.g. ≥1.01 times, ≥1.02 times, ≥1.03 times, ≥1.04 times, ≥1.05 times, ≥1.06 times, ≥1.07 times, ≥1.08 times, ≥1.09 times, ≥1.1 times, ≥1.2 times, ≥1.3 times, 1.4 times, ≥1.5 times, ≥1.6 times, ≥1.7 times, ≥1.8 times, ≥1.9 times, ≥2 times, ≥2.1 times, ≥2.2 times, 2.3 times, ≥2.4 times, ≥2.5 times, ≥2.6 times, ≥2.7 times, ≥2.8 times, ≥2.9 times, ≥3 times, ≥3.5 times, 4 times, ≥4.5 times, ≥5 times, ≥6 times, ≥7 times, ≥8 times, ≥9 times, ≥10 times, ≥15 times, ≥20 times, ≥25 times, ≥30 times, ≥35 times, ≥40 times, ≥45 times, ≥50 times, ≥60 times, ≥70 times, ≥80 times, ≥90 times, ≥100 times, ≥200 times, ≥300 times, ≥400 times, ≥500 times, ≥600 times, ≥700 times, ≥800 times, ≥900 times, ≥1000 times the level of cell proliferation/population expansion, growth factor (e.g. IL-2) expression, cytotoxic/effector factor (e.g. IFNγ, granzyme, perforin, granulysin, CD107a, TNFα, FASL) expression or cytotoxic activity displayed by comparable cells not comprising/expressing the polypeptide, nucleic acid/plurality of nucleic acids or expression vector/plurality of expression vectors according to the present disclosure, under the same conditions.

Reduced susceptibility to cell killing may be determined by detection of a level of cell killing which is less than 1 times, e.g. one of ≤0.99 times, ≤0.95 times, ≤0.9 times, ≤0.85 times, ≤0.8 times, ≤0.85 times, ≤0.75 times, ≤0.7 times, ≤0.65 times, ≤0.6 times, ≤0.55 times, ≤0.5 times, ≤0.45 times, ≤0.4 times, ≤0.35 times, 0.3 times, ≤0.25 times, ≤0.2 times, ≤0.15 times, ≤0.1 times, ≤0.09 times, ≤0.08 times, ≤0.07 times, ≤0.06 times, ≤0.05 times, ≤0.04 times, ≤0.03 times, ≤0.02 times or ≤0.01 times the level of cell killing of comparable cells not comprising/expressing the polypeptide, nucleic acid/plurality of nucleic acids or expression vector/plurality of expression vectors according to the present disclosure, under the same conditions.

In some embodiments of the present disclosure wherein the cell comprising/expressing a polypeptide, a nucleic acid/plurality of nucleic acids or an expression vector/plurality of expression vectors according to the present disclosure is a virus-specific T cell, the cell may display increased cell proliferation/population expansion, growth factor (e.g. IL-2) expression, cytotoxic/effector factor (e.g. IFNγ, granzyme, perforin, granulysin, CD107a, TNFα, FASL) expression and/or cytotoxic activity following exposure to a cell comprising/expressing a TCR specific for an MHC complex presented by the cell, as compared to the level displayed by a cell comprising/expressing the same polypeptide/nucleic acid/plurality of nucleic acids/expression vector/plurality of expression vectors which is not a virus-specific T cell. In some embodiments of the present disclosure wherein the cell comprising/expressing a polypeptide, a nucleic acid/plurality of nucleic acids or an expression vector/plurality of expression vectors according to the present disclosure is a virus-specific T cell, the cell may display reduced susceptibility to cell killing by a cell comprising/expressing a TCR specific for an MHC complex presented by the cell, as compared to susceptibility to cell killing displayed by a cell comprising/expressing the same polypeptide/nucleic acid/plurality of nucleic acids/expression vector/plurality of expression vectors which is not a virus-specific T cell.

In some embodiments the cell which is not a virus-specific T cell may be a naïve T cell, a non-activated T cell, or a T cell activated by treatment with agonist anti-CD3 antibody and/or agonist anti-CD28 antibody.

A naïve T cell is a T cell which has not encountered peptide or MHC-peptide complex for which the TCR of the T cell has high affinity, e.g. presented by an APC. A non-activated T cell is a T cell which has not undergone the process of T cell activation. In some embodiments a non-activated T cell is a T cell which has not encountered MHC-peptide complex for which the TCR of the T cell has high affinity in the context of a positive costimulatory signal from an APC. In some embodiments, a non-activated T cell is a T cell which has not been stimulated through CD3, optionally in combination with stimulation through CD28, optionally in the presence of mitogenic cytokine(s) (e.g. IL-2). A T cell activated by treatment with agonist anti-CD3 antibody and/or agonist anti-CD28 antibody may have been activated in vitro or ex vivo by stimulation through treatment with agonist anti-CD3 antibody and/or agonist anti-CD28 antibody, optionally in the presence of mitogenic cytokine(s) (e.g. IL-2).

The increased cell proliferation/population expansion, growth factor (e.g. IL-2) expression, cytotoxic/effector factor (e.g. IFNγ, granzyme, perforin, granulysin, CD107a, TNFα, FASL) expression or cytotoxic activity may be one of greater than 1 times, e.g. ≥1.01 times, ≥1.02 times, ≥1.03 times, ≥1.04 times, ≥1.05 times, ≥1.06 times, ≥1.07 times, ≥1.08 times, ≥1.09 times, ≥1.1 times, ≥1.2 times, ≥1.3 times, 1.4 times, ≥1.5 times, ≥1.6 times, ≥1.7 times, ≥1.8 times, ≥1.9 times, ≥2 times, ≥2.1 times, ≥2.2 times, 2.3 times, ≥2.4 times, ≥2.5 times, ≥2.6 times, ≥2.7 times, ≥2.8 times, ≥2.9 times, ≥3 times, ≥3.5 times, ≥4 times, ≥4.5 times, ≥5 times, ≥6 times, ≥7 times, ≥8 times, ≥9 times, ≥10 times, ≥15 times, ≥20 times, ≥25 times, ≥30 times, ≥35 times, ≥40 times, ≥45 times, ≥50 times, ≥60 times, ≥70 times, ≥80 times, ≥90 times, ≥100 times, ≥200 times, ≥300 times, ≥400 times, ≥500 times, ≥600 times, ≥700 times, ≥800 times, ≥900 times, ≥1000 times the level of cell proliferation/population expansion, growth factor (e.g. IL-2) expression, cytotoxic/effector factor (e.g. IFNγ, granzyme, perforin, granulysin, CD107a, TNFα, FASL) expression or cytotoxic activity displayed by cells comprising/expressing the same polypeptide/nucleic acid/plurality of nucleic acids/expression vector/plurality of expression vectors which are not virus-specific T cells (e.g. which are naïve T cells, non-activated T cells, or T cells activated by treatment with agonist anti-CD3 antibody and/or agonist anti-CD28 antibody), under the same conditions.

Reduced susceptibility to cell killing may be determined by detection of a level of cell killing which is less than 1 times, e.g. one of ≤0.99 times, ≤0.95 times, ≤0.9 times, ≤0.85 times, ≤0.8 times, ≤0.85 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.09 times, ≤0.08 times, ≤0.07 times, ≤0.06 times, ≤0.05 times, ≤0.04 times, ≤0.03 times, ≤0.02 times or ≤0.01 times the level of cell killing of cells comprising/expressing the same polypeptide/nucleic acid/plurality of nucleic acids/expression vector/plurality of expression vectors which are not virus-specific T cells (e.g. which are naïve T cells, non-activated T cells, or T cells activated by treatment with agonist anti-CD3 antibody and/or agonist anti-CD28 antibody), under the same conditions.

In some embodiments—particularly embodiments wherein the nucleic acid/plurality of nucleic acids comprises a control element for inducible upregulation of expression of the polypeptide and/or wherein the nucleic acid/plurality of nucleic acids encodes a conditional expression system for controlling expression of the polypeptide—cells comprising a nucleic acid/plurality of nucleic acids or an expression vector/plurality of expression vectors according to the present disclosure may display a level of cell proliferation/population expansion which is similar to the level of proliferation/expansion by comparable cells not comprising a nucleic acid/plurality of nucleic acids or an expression vector/plurality of expression vectors according to the present disclosure (in the absence of agent for inducing expression of the polypeptide).

A level of cell proliferation/population expansion which is similar to the level of cell proliferation/population by a reference cell or population of cells may be ≥0.2 times and ≤5 times, e.g. ≥0.3 times and ≤4 times, ≥0.4 times and ≤3 times, ≥0.5 times and ≤2 times, ≥0.6 times and ≤1.75 times, ≥0.7 times and ≤1.5 times, ≥0.75 times and ≤1.25 times, ≥0.8 times and ≤1.2 times, ≥0.85 times and ≤1.15 times, ≥0.9 times and ≤1.1 times, ≥0.91 times and ≤1.09 times, ≥0.92 times and ≤1.08 times, ≥0.93 times and ≤1.07 times, ≥0.94 times and ≤1.06 times, ≥0.95 times and ≤1.05 times, ≥0.96 times and ≤1.04 times, ≥0.97 times and ≤1.03 times, ≥0.98 times and ≤1.02 times, or ≥0.99 times and ≤1.01 times the level of cell proliferation/population expansion displayed by the reference cell or population of cells, as determined in an appropriate assay.

In some embodiments, cells comprising a nucleic acid/plurality of nucleic acids or an expression vector/plurality of expression vectors according to the present disclosure wherein the nucleic acid/plurality of nucleic acids comprises a control element for inducible upregulation of expression of the polypeptide may display (in the absence of agent for inducing expression of the polypeptide) increased cell proliferation/population expansion and/or reduced fratricide as compared to comparable cells that constitutively express the polypeptide of the disclosure. Herein, “fratricide” refers to cell killing of like cells (e.g. cell killing of autogeneic cells). Advantageously, such cells are readily expanded (e.g. in in vitro or ex vivo culture or in vivo), and as such are associated with advantages for generating/expanding populations of cells comprising a nucleic acid/plurality of nucleic acids or an expression vector/plurality of expression vectors according to the present disclosure.

The increased cell proliferation/population expansion may be one of greater than 1 times, e.g. ≥1.01 times, ≥1.02 times, ≥1.03 times, ≥1.04 times, ≥1.05 times, ≥1.06 times, ≥1.07 times, ≥1.08 times, ≥1.09 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥2.1 times, ≥2.2 times, ≥2.3 times, ≥2.4 times, ≥2.5 times, ≥2.6 times, ≥2.7 times, ≥2.8 times, ≥2.9 times, ≥3 times, ≥3.5 times, ≥4 times, ≥4.5 times, ≥5 times, ≥6 times, ≥7 times, ≥8 times, ≥9 times, ≥10 times, ≥15 times, ≥20 times, ≥25 times, ≥30 times, ≥35 times, ≥40 times, ≥45 times, ≥50 times, ≥60 times, ≥70 times, ≥80 times, ≥90 times, ≥100 times, ≥200 times, ≥300 times, ≥400 times, ≥500 times, ≥600 times, ≥700 times, ≥800 times, ≥900 times, ≥1000 times the level of cell proliferation/population expansion displayed by comparable cells that constitutively express the polypeptide of the disclosure, as determined in an appropriate assay.

A reduced level of fratricide may be a level of fratricide which is less than 1 times, e.g. one of ≤0.99 times, ≤0.95 times, ≤0.9 times, ≤0.85 times, ≤0.8 times, ≤0.85 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.09 times, ≤0.08 times, ≤0.07 times, ≤0.06 times, ≤0.05 times, ≤0.04 times, ≤0.03 times, ≤0.02 times or ≤0.01 times the level of fratricide displayed by comparable cells that constitutively express the polypeptide of the disclosure, as determined in an appropriate assay.

### Compositions

The present disclosure also provides compositions comprising a polypeptide, a nucleic acid or a plurality of nucleic acids, an expression vector or a plurality of expression vectors, or a cell according to the disclosure.

The polypeptide, nucleic acid/plurality of nucleic acids, expression vector/plurality of expression vectors or cell according to the present disclosure may be formulated as pharmaceutical compositions for clinical use and may comprise a pharmaceutically acceptable carrier, diluent, excipient or adjuvant.

In accordance with the present disclosure methods are also provided for the production of pharmaceutically useful compositions, such methods of production may comprise one or more steps selected from: isolating a polypeptide, a nucleic acid/plurality of nucleic acids, an expression vector/plurality of expression vectors, or a cell as described herein; and/or mixing a polypeptide, a nucleic acid/plurality of nucleic acids, an expression vector/plurality of expression vectors, or a cell as described herein with a pharmaceutically acceptable carrier, adjuvant, excipient or diluent.

For example, a further aspect of the present disclosure relates to a method of formulating or producing a medicament or pharmaceutical composition, the method comprising formulating a pharmaceutical composition or medicament by mixing a polypeptide, a nucleic acid/plurality of nucleic acids, an expression vector/plurality of expression vectors, or a cell as described herein with a pharmaceutically acceptable carrier, adjuvant, excipient or diluent.

The present disclosure also provides a kit of parts comprising one or more of a polypeptide, a nucleic acid/plurality of nucleic acids, an expression vector/plurality of expression vectors, a cell or a composition according to the present disclosure.

In some embodiments the kit may have at least one container having a predetermined quantity of a polypeptide, a nucleic acid/plurality of nucleic acids, an expression vector/plurality of expression vectors, a cell or a composition according to the disclosure or a composition according to the present disclosure.

The polypeptide, nucleic acid/plurality of nucleic acids, expression vector/plurality of expression vectors, cell or composition with instructions for administration to a patient in order to treat/prevent a specified disease/condition. The polypeptide, nucleic acid/plurality of nucleic acids, expression vector/plurality of expression vectors, cell or composition may be formulated so as to be suitable for injection or infusion to the blood, a particular site, a tissue, an organ or a tumor.

In some embodiments the kit may comprise materials for producing a cell according to the present disclosure. For example, the kit may comprise materials for modifying a cell to express or comprise a polypeptide, a nucleic acid/plurality of nucleic acids, an expression vector/plurality of expression vectors according to the present disclosure, or materials for introducing into a cell a nucleic acid/plurality of nucleic acids or an expression vector/plurality of expression vectors according to the present disclosure.

The kit may comprise materials for producing an immune cell specific for a virus; for example, the kit may comprise pepmixes of one or more viral antigens.

In some embodiments the kit may further comprise at least one container having a predetermined quantity of another therapeutic/prophylactic agent (e.g. an anti-infective agent or chemotherapy agent). In such embodiments, the kit may also comprise a second medicament or pharmaceutical composition such that the two medicaments or pharmaceutical compositions may be administered simultaneously or separately such that they provide a combined treatment.

### Methods Using the Articles of the Present Disclosure

The articles of the present disclosure are useful in methods to reduce/prevent alloreactive immune responses (particularly T cell-mediated alloreactive immune responses) and the deleterious consequences thereof.

The articles of the present disclosure can be used in methods involving allotransplantation, e.g. to treat/prevent a disease/condition in a subject. As used herein, “allotransplantation” refers to the transplantation to a recipient subject of cells, tissues or organs which are genetically non-identical to the recipient subject. The cells, tissues or organs may be from, or may be derived from, cells, tissues or organs of a donor subject that is genetically non-identical to the recipient subject. Allotransplantation is distinct from autotransplantation, which refers to the transplantation of cells, tissues or organs which are genetically identical to the recipient subject.

Cells, tissues and organs which may be allotransplanted include e.g. immune cells, the heart, lung, kidney, liver, pancreas, intestine, face, cornea, skin, hematopoietic stem cells (bone marrow), blood, hands, leg, penis, bone, uterus, thymus, islets of Langerhans, heart valve and ovary. A population of cells, tissue or organ to be allotransplanted may be referred to as an allotransplant.

Transplantation of immune cells may also be referred to as adoptive cell transfer (ACT). Adoptive cell transfer (ACT) generally refers to a process by which immune cells are obtained from a subject, typically by drawing a blood sample from which the immune cells are isolated. The immune cells are then typically treated or altered in some way and/or expanded, and then administered either to the same subject (in the case of adoptive transfer of autologous cells) or to a different subject (in the case of adoptive transfer of allogeneic cells). The adoptive cell transfer is typically aimed at providing an immune cell population with certain desired characteristics to a subject, or increasing the frequency of immune cells with such characteristics in that subject. Adoptive transfer of T cells is described, for example, in Kalos and June 2013, Immunity 39(1): 49-60, which is hereby incorporated by reference in its entirety.

The articles of the present disclosure are particularly useful in methods comprising adoptive cell transfer of allogeneic cells. Cells expressing/comprising the polypeptides, nucleic acid(s) or expression vector(s) of the present disclosure are less susceptible to T cell mediated alloreactive immune responses of the recipient following adoptive transfer, and thus exhibit enhanced proliferation/survival in the recipient after transfer. Thus the polypeptides, nucleic acid(s), expression vector(s), cells and compositions of the present disclosure can be used to enhance the therapeutic/prophylactic utility of adoptively transferred allogeneic cells.

In some embodiments the adoptive transfer is of T cells, e.g. CD3+ T cells. In some embodiments, the T cells are CD3+, CD4+ T cells. In some embodiments, the T cells are CD3+, CD8+ T cells. In some embodiments, the T cells are T helper cells (TH cell)). In some embodiments, the T cells are cytotoxic T cells (e.g. a cytotoxic T lymphocytes (CTLs)). In some embodiments, the T cells are virus-specific T cells, e.g. virus-specific T cells as described herein. In some embodiments, T cells are specific for EBV, HPV, HBV, HCV or HIV.

The disease/condition to be treated/prevented by the adoptive cell transfer can be any disease/condition which would derive therapeutic or prophylactic benefit from an increase in the number of the adoptively transferred cells. In some embodiments the disease/condition is a T cell dysfunctional disorder, an infectious disease or a cancer.

A T cell dysfunctional disorder may be a disease/condition in which normal T cell function is impaired causing downregulation of the subject's immune response to pathogenic antigens, e.g. generated by infection by exogenous agents such as microorganisms, bacteria and viruses, or generated by the host in some disease states such as in some forms of cancer (e.g. in the form of tumor-associated antigens).

The T cell dysfunctional disorder may comprise T cell exhaustion or T cell anergy. T cell exhaustion comprises a state in which CD8+ T cells fail to proliferate or exert T cell effector functions such as cytotoxicity and cytokine (e.g. IFNγ) secretion in response to antigen stimulation. Exhausted T cells may also be characterised by sustained expression of one or more markers of T cell exhaustion, e.g. PD-1, CTLA-4, LAG-3, TIM-3. The T cell dysfunctional disorder may be manifest as an infection, or inability to mount an effective immune response against an infection. The infection may be chronic, persistent, latent or slow, and may be the result of bacterial, viral, fungal or parasitic infection. As such, treatment may be provided to patients having a bacterial, viral or fungal infection. Examples of bacterial infections include infection with Helicobacter pylori. Examples of viral infections include infection with HIV, hepatitis B or hepatitis C. The T cell dysfunctional disorder may be associated with a cancer, such as tumor immune escape. Many human tumors express tumor-associated antigens recognised by T cells and capable of inducing an immune response.

An infectious disease may be e.g. bacterial, viral, fungal, or parasitic infection. In some embodiments it may be particularly desirable to treat chronic/persistent infections, e.g. where such infections are associated with T cell dysfunction or T cell exhaustion. It is well established that T cell exhaustion is a state of T cell dysfunction that arises during many chronic infections (including viral, bacterial and parasitic), as well as in cancer (Wherry Nature Immunology Vol. 12, No. 6, p 492-499, June 2011). Examples of bacterial infections that may be treated include infection by Bacillus spp., Bordetella pertussis, Clostridium spp., Corynebacterium spp., Vibrio chloerae, Staphylococcus spp., Streptococcus spp. Escherichia, Klebsiella, Proteus, Yersinia, Erwina, Salmonella, Listeria sp, Helicobacter pylori, mycobacteria (e.g. Mycobacterium tuberculosis) and Pseudomonas aeruginosa. For example, the bacterial infection may be sepsis or tuberculosis. Examples of viral infections that may be treated include infection by influenza virus, measles virus, hepatitis B virus (HBV), hepatitis C virus (HCV), human immunodeficiency virus (HIV), lymphocytic choriomeningitis virus (LCMV), Herpes simplex virus and human papilloma virus (HPV). Examples of fungal infections that may be treated include infection by Alternaria sp, Aspergillus sp, Candida sp and Histoplasma sp. The fungal infection may be fungal sepsis or histoplasmosis. Examples of parasitic infections that may be treated include infection by Plasmodium species (e.g. Plasmodium falciparum, Plasmodium yoeli, Plasmodium ovale, Plasmodium vivax, or Plasmodium chabaudi chabaudi). The parasitic infection may be a disease such as malaria, leishmaniasis and toxoplasmosis.

In particular embodiments, the disease/condition to be treated/prevented is a cancer. The cancer may be any unwanted cell proliferation (or any disease manifesting itself by unwanted cell proliferation), neoplasm or tumor or increased risk of or predisposition to the unwanted cell proliferation, neoplasm or tumor. The cancer may be benign or malignant and may be primary or secondary (metastatic). A neoplasm or tumor may be any abnormal growth or proliferation of cells and may be located in any tissue.

Examples of tissues include the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, white blood cells. In some embodiments, the cancer to be treated may be a cancer of a tissue selected from the group consisting of colon, rectum, nasopharynx, cervix, oropharynx, stomach, liver, head and neck, oral cavity, oesophagus, lip, mouth, tongue, tonsil, nose, throat, salivary gland, sinus, pharynx, larynx, prostate, lung, bladder, skin, kidney, ovary or mesothelium.

Tumors to be treated may be nervous or non-nervous system tumors. Nervous system tumors may originate either in the central or peripheral nervous system, e.g. glioma, medulloblastoma, meningioma, neurofibroma, ependymoma, Schwannoma, neurofibrosarcoma, astrocytoma and oligodendroglioma. Non-nervous system cancers/tumors may originate in any other non-nervous tissue, examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T cell lymphoma (CTCL), chronic lymphocytic leukemia (CLL), hepatoma, epidermoid carcinoma, prostate carcinoma, breast cancer, lung cancer, colon cancer, ovarian cancer, pancreatic cancer, thymic carcinoma, NSCLC, haematologic cancer and sarcoma.

In some embodiments the cancer is selected from the group consisting of: colon cancer, colon carcinoma, colorectal cancer, nasopharyngeal carcinoma, cervical carcinoma, oropharyngeal carcinoma, gastric carcinoma, hepatocellular carcinoma, head and neck cancer, head and neck squamous cell carcinoma (HNSCC), oral cancer, laryngeal cancer, prostate cancer, lung cancer, small cell lung cancer, non-small cell lung cancer, bladder cancer, urothelial carcinoma, melanoma, advanced melanoma, renal cell carcinoma, ovarian cancer or mesothelioma. The cancer may be positive for one or more particular antigens.

In some embodiments the cancer to be treated/prevented is a virus-associated cancer, e.g. an EBV-associated cancer or a HPV-associated cancer. “EBV associated” and “HPV associated” cancers may be a cancers which are caused or exacerbated by infection with the respective viruses, cancers for which infection is a risk factor and/or cancers for which infection is positively associated with onset, development, progression, severity or metastasis. EBV-associated cancers which may be treated with cells produced by methods of the disclosure include nasopharyngeal carcinoma (NPC) and gastric carcinoma (GC). HPV-associated medical conditions that may be treated with cells produced by methods of the disclosure include at least dysplasias of the genital area(s), cervical intraepithelial neoplasia, vulvar intraepithelial neoplasia, penile intraepithelial neoplasia, anal intraepithelial neoplasia, cervical cancer, anal cancer, vulvar cancer, vaginal cancer, penile cancer, genital cancers, oral papillomas, oropharyngeal cancer. In some embodiments, the cancer to be treated in accordance with various aspects of the present disclosure is one or more of lymphoma (e.g. Epstein-Barr Virus (EBV)-positive lymphoma), nasopharyngeal carcinoma (NPC; e.g. EBV-positive NPC), cervical carcinoma (CC; e.g. human papillomavirus (HPV)-positive CC), oropharyngeal carcinoma (OPC; e.g. HPV-positive OPC), gastric carcinoma (GC; e.g. EBV-positive GC), hepatocellular carcinoma (HCC; e.g. Hepatitis B Virus (HBV)-positive HCC), lung cancer (e.g. non-small cell lung cancer (NSCLC)) and head and neck cancer (e.g. cancer originating from tissues of the lip, mouth, nose, sinuses, pharynx or larynx, e.g. head and neck squamous cell carcinoma (HNSCC)).

Modification of immune cells to be adoptively transferred in a method of allogeneic ACT to express or comprise a polypeptide, a nucleic acid or plurality of nucleic acids, or an expression vector or plurality of expression vectors according to the present disclosure can be considered to be a modification to improve the persistence/survival of the allogeneic cells in the recipient subject.

In some embodiments, methods of treating or preventing a disease/condition comprising adoptive transfer of allogeneic immune cells according to the present disclosure comprise:
(a) isolating immune cells from a subject;
(b) modifying at least one immune cell to express or comprise a polypeptide, a nucleic acid or plurality of nucleic acids, or an expression vector or plurality of expression vectors according to the present disclosure;
(c) optionally expanding the modified at least one immune cell, and;
(d) administering the modified at least one immune cell to a subject.

In some embodiments the method steps for production of cells comprising/expressing a polypeptide, a nucleic acid/plurality of nucleic acids or an expression vector/plurality of expression vectors according to the present disclosure may comprise one or more of: isolating immune cells from a subject; taking a blood sample from a subject; isolating PBMCs from a blood sample; modifying at least one immune cell to express or comprise a polypeptide, a nucleic acid or plurality of nucleic acids, or an expression vector or plurality of expression vectors according to the present disclosure, e.g. by introducing a nucleic acid/plurality of nucleic acids or an expression vector/plurality of expression vectors according to the present disclosure into at least one immune cell; culturing the modified at least one immune cell in in vitro or ex vivo cell culture; generating or expanding a population of modified immune cells; inducing expression of the polypeptide e.g. using a control element and/or a conditional expression system; collecting the modified immune cells; mixing the modified immune cells with an adjuvant, diluent, or carrier; administering the modified immune cells to a subject.

In some particular embodiments the methods comprise adoptive transfer of allogeneic immune cells specific for a virus. In some embodiments the methods comprise:
(a) isolating immune cells from a subject;
(b) generating or expanding a population of immune cells specific for a virus by a method comprising: stimulating the immune cells by culture in the presence of antigen presenting cells (APCs) presenting one or more peptides of the virus;
(c) modifying at least one immune cell specific for a virus to express or comprise a polypeptide, a nucleic acid or plurality of nucleic acids, or an expression vector or plurality of expression vectors according to the present disclosure;
(d) optionally expanding the modified at least one immune cell specific for a virus, and;
(e) administering the modified at least one immune cell specific for a virus to a subject.

In some embodiments the methods comprise one or more of: isolating immune cells from a subject; taking a blood sample from a subject; isolating PBMCs from a blood sample; generating/expanding a population of immune cells specific for a virus (e.g. by culturing PBMCs in the presence of cells (e.g. APCs) comprising/expressing antigen(s)/peptide(s) of the virus); culturing immune cells specific for a virus in in vitro or ex vivo cell culture; collecting immune cells specific for a virus; modifying at least one immune cell specific for a virus to express or comprise a polypeptide, a nucleic acid or plurality of nucleic acids, or an expression vector or plurality of expression vectors according to the present disclosure, e.g. by introducing a nucleic acid/plurality of nucleic acids or an expression vector/plurality of expression vectors according to the present disclosure into at least one immune cell specific for a virus; culturing the modified at least one immune cell specific for a virus in in vitro or ex vivo cell culture; generating or expanding a population of modified immune cells specific for a virus; collecting the modified immune cells specific for a virus; mixing the modified immune cells specific for a virus with an adjuvant, diluent, or carrier; administering the modified immune cells specific for a virus to a subject.

The present disclosure further provides a method of depleting a population of immune cells of alloreactive immune cells (e.g. alloreactive T cells), comprising:
(a) modifying at least one immune cell from a first subject to express or comprise a polypeptide, a nucleic acid or plurality of nucleic acids, or an expression vector or plurality of expression vectors according to the present disclosure; and
(b) contacting a population of immune cells to be depleted of alloreactive immune cells (e.g. alloreactive T cells) from a second, allogeneic subject with the modified at least one immune cell.

The population of immune cells to be depleted of (or reduced in quantity of, such as 2-fold, 10-fold, 100-fold, 1000-fold, 10000-fold, and so forth) alloreactive immune cells may have been isolated from a subject or may be in situ (i.e. in the second, allogeneic subject). Modification of the at least one immune cell from the first subject may be performed in vitro or ex vivo, or in vivo in the first subject. Contacting of the population of immune cells to be depleted of alloreactive immune cells from a second, allogeneic subject with the modified at least one immune cell may be performed in vitro or ex vivo, or in vivo in the second, allogeneic subject or in the first subject. Method steps performed in vitro or ex vivo may comprise in vitro or ex vivo cell culture.

In some embodiments the methods comprise one or more of: isolating immune cells from the first subject and/or the second, allogeneic subject; taking a blood sample from the first subject and/or the second, allogeneic subject; isolating PBMCs from blood sample(s); culturing immune cells from the first subject and/or the second, allogeneic subject in in vitro or ex vivo cell culture; modifying at least one immune cell from the first subject to express or comprise a polypeptide, a nucleic acid or plurality of nucleic acids, or an expression vector or plurality of expression vectors according to the present disclosure, e.g. by introducing a nucleic acid/plurality of nucleic acids or an expression vector/plurality of expression vectors according to the present disclosure into the at least one immune cell; culturing the modified at least one immune cell specific in in vitro or ex vivo cell culture; generating or expanding a population of modified immune cells; collecting the modified immune cells; generating or expanding a population of immune cells to be depleted of alloreactive immune cells; contacting a population of immune cells to be depleted of alloreactive immune cells from a second, allogeneic subject with the modified at least one immune cell, e.g. in in vitro or ex vivo cell culture; collecting the population of cells depleted of alloreactive immune cells.

The articles and methods of the present disclosure are particularly useful in methods involving allotransplantation, and also in the processing/production of allotransplants. In particular, the present articles and methods are contemplated for use in the production and administration of “off-the-shelf” materials for use in therapeutic and prophylactic methods comprising administration of allogeneic material.

As demonstrated herein, immune cells comprising/expressing polypeptides, nucleic acid(s) or expression vector(s) according to the present disclosure can be used to deplete alloreactive T cells in the recipient for an allotransplant, which could otherwise cause/promote graft rejection.

Conversely, immune cells obtained/derived from the recipient for an allotransplantation can be engineered to comprise/express the polypeptides, nucleic acid(s) or expression vector(s) according to the present disclosure, and used to deplete alloreactive cells within populations of cells, tissues and/or organs to be transplanted, which could otherwise lead to graft versus host disease (GVHD).

Thus immune cells comprising/expressing polypeptides, nucleic acid(s) or expression vector(s) according to the present disclosure are useful as agents to enhance the effectiveness of allotransplantation of cells, tissues and/organs.

Accordingly, the present disclosure provides methods for the treatment/prevention of diseases/conditions caused or exacerbated by alloreactive immune cells associated with allotransplantation. Such diseases/conditions include graft rejection and GVHD, which are described in detail in Perkey and Maillard Annu Rev Pathol. (2018) 13:219-245, which is hereby incorporated by reference in its entirety.

Graft rejection refers to the destruction of transplanted cells/tissue/organs by a recipient's immune system following transplantation. Where graft rejection is of an allotransplant, it may be referred to as allograft rejection. Graft-versus-host disease (GVHD) can occur following allotransplantation of large numbers of donor immune cells, and involves reactivity of donor-derived immune cells against allogeneic recipient cells/tissues/organs.

The present disclosure provides methods of treating/preventing graft rejection following allotransplantation, comprising administering at least one immune cell of the donor subject for the allotransplant modified to express or comprise a polypeptide, a nucleic acid or plurality of nucleic acids, or an expression vector or plurality of expression vectors of the present disclosure to the recipient subject for the allotransplant.

The aim for such methods is to reduce/remove the ability of the receipt subject to mount an alloreactive immune response to the allotransplant. The donor immune cells comprising/expressing polypeptides, nucleic acid(s) or expression vector(s) according to the present disclosure are useful to eliminate immune cells in the recipient that would otherwise effect an alloreactive immune response against donor cells, tissue and/or organs, through effector activity triggered by ligation of the MHC class I α:CHAR complex by alloreactive T cells.

In some embodiments the methods comprise administering a plurality of (i.e. a population of) donor subject immune cells modified to express or comprise a polypeptide, a nucleic acid or plurality of nucleic acids, or an expression vector or plurality of expression vectors of the present disclosure to the recipient subject for the allotransplant. It will be appreciated that instead of immune cell(s) of the donor subject, immune cells which are autogeneic to the donor could be used in such methods.

In some embodiments administration of the modified at least one immune cell of the donor subject and allotransplantation are performed simultaneously, i.e. at the same time, or within e.g. 1 hr, 2 hrs, 3 hrs, 4 hrs, 5 hrs, 6 hrs, 8 hrs, 12 hrs, 24 hrs, 36 hrs or 48 hrs. In some embodiments administration of the modified at least one immune cell of the donor subject and allotransplantation are performed sequentially. The time interval between administration and allotransplantation may be any time interval, including hours, days, weeks, months, or years.

In some embodiments the modified at least one immune cell of the donor subject is administered to the recipient prior to allotransplantation. In some embodiments the modified at least one immune cell of the donor subject is administered to the recipient after allotransplantation.

In some embodiments the methods comprise additional intervention to treat/prevent alloreactivity and/or graft rejection. In some embodiments the methods to treat/prevent alloreactivity and/or graft rejection comprise administration of immunosuppressive therapy such as treatment with corticosteroids (e.g. prednisolone, hydrocortisone), calcineurin inhibitors (e.g. cyclosporin, tacrolimus) anti-proliferative agents (e.g. azathioprinem, mycophenolic acid) and/or mTOR inhibitors (e.g. sirolimus, everolimus). In some embodiments the methods to treat/prevent alloreactivity and/or graft rejection comprise antibody therapy, such as treatment with monoclonal anti-IL-2Ra receptor antibodies (e.g. basiliximab, daclizumab), anti-T cell antibodies (e.g. anti-thymocyte globulin, anti-lymphocyte globulin) and/or anti-CD20 antibodies (e.g. rituximab). In some embodiments the methods to treat/prevent alloreactivity and/or graft rejection comprise blood transfusion and/or bone marrow transplantation.

The present disclosure also provides methods of treating/preventing graft versus host disease (GVHD) associated with allotransplantation, comprising contacting the allotransplant with at least one immune cell of the recipient subject for the allotransplant modified to express or comprise a polypeptide, a nucleic acid or plurality of nucleic acids, or an expression vector or plurality of expression vectors of the present disclosure. The allotransplant may then be administered to the recipient subject.

The aim for such methods is to eliminate alloreactive immune cells (e.g. alloreactive T cells) in the allotransplant. The recipient immune cells comprising/expressing polypeptides, nucleic acid(s) or expression vector(s) according to the present disclosure are useful to eliminate immune cells in the allotransplant that would otherwise effect an alloreactive immune response against cells, tissue and/or organs of the recipient, through effector activity triggered by ligation of the MHC class I α:CHAR complex by alloreactive T cells.

In some embodiments the methods comprise contacting the allotransplant with a plurality of (i.e. a population of) recipient immune cells modified to express or comprise a polypeptide, a nucleic acid or plurality of nucleic acids, or an expression vector or plurality of expression vectors of the present disclosure. It will be appreciated that instead of immune cell(s) of the recipient subject, immune cells which are autogeneic to the recipient could be used in such methods.

In some embodiments the methods comprise additional intervention to treat/prevent alloreactivity and/or GVHD. In some embodiments the methods to treat/prevent alloreactivity and/or GVHD comprise administration of immunosuppressive therapy such as treatment with glucocorticoids (e.g. prednisone), calcineurin inhibitors (e.g. cyclosporin, tacrolimus), anti-proliferative agents (e.g. azathioprinem, mycophenolic acid) and/or mTOR inhibitors (e.g. sirolimus, everolimus). In some embodiments the methods to treat/prevent alloreactivity and/or GVHD comprise antibody therapy, such as treatment with monoclonal anti-IL-2Rα receptor antibodies (e.g. basiliximab, daclizumab), anti-T cell antibodies (e.g. anti-thymocyte globulin, anti-lymphocyte globulin) and/or anti-CD20 antibodies (e.g. rituximab). In some embodiments the methods to treat/prevent alloreactivity and/or GVHD comprise blood transfusion and/or bone marrow transplantation.

In a further aspect the present disclosure provides methods for treating/preventing autoimmune diseases/conditions, and methods for depleting populations of cells of autoreactive immune cells (e.g. autoreactive T cells). Such methods employ cells expressing/comprising a polypeptide, a nucleic acid or plurality of nucleic acids, or an expression vector or plurality of expression vectors according to the present disclosure, additionally comprising/expressing an autoantigenic peptide:MHC class I α polypeptide complex.

The CHAR of the present disclosure associates with the autoantigenic peptide:MHC class I α polypeptide complex at the cell surface, and upon engagement by an autoreactive T cell, the cell expressing the CHAR exhibits cytotoxicity to the autoreactive T cell.

Accordingly, the present disclosure provides a method of depleting (or reducing in quantity) a population of immune cells of autoreactive immune cells (e.g. autoreactive T cells), comprising:
(a) modifying at least one immune cell comprising/expressing an autoantigenic peptide:MHC class I α polypeptide complex to express or comprise a polypeptide, a nucleic acid or plurality of nucleic acids, or an expression vector or plurality of expression vectors according to the present disclosure; and
(b) contacting a population of immune cells to be depleted of autoreactive immune cells (e.g. autoreactive T cells) with the modified at least one immune cell.

As used herein, an “autoantigenic peptide” refers to a peptide of an autoantigen. An autoantigen is a factor produced by a subject (e.g. a protein) against which the subject's immune system initiates an immune response in autoimmunity. An autoantigenic peptide:MHC class I α polypeptide complex refers to a polypeptide complex comprising an autoantigenic peptide and MHC class I α polypeptide.

The population of immune cells to be depleted of autoreactive immune cells may have been isolated from a subject or may be in situ (i.e. in the subject). The immune cell comprising/expressing an autoantigenic peptide:MHC class I α polypeptide complex may do as a consequence of endogenous expression of the autoantigen, or as a consequence of having been modified to do so (e.g. modification to comprise/express nucleic acid encoding the autoantigen/fragment thereof, modification to upregulate expression of the autoantigen/fragment thereof, or through being pulsed with the autoantigen/peptide(s) thereof). Modification of the immune cell may be performed in vitro or ex vivo, or in vivo in the subject. Contacting of the population of immune cells to be depleted of autoreactive immune cells from the subject with the modified at least one immune cell may be performed in vitro or ex vivo, or in vivo in the subject. Method steps performed in vitro or ex vivo may comprise in vitro or ex vivo cell culture.

In some embodiments the methods comprise one or more of: isolating immune cells from a subject; taking a blood sample from a subject; isolating PBMCs from blood sample(s); culturing immune cells from a subject in in vitro or ex vivo cell culture; modifying at least one immune cell to comprise/express an autoantigenic peptide:MHC class I α polypeptide complex, e.g. by modification to comprise/express nucleic acid encoding an autoantigen/fragment thereof, modification to upregulate expression of the autoantigen/fragment thereof, or through being pulsed with the autoantigen/peptide(s) thereof; modifying at least one immune cell to express or comprise a polypeptide, a nucleic acid or plurality of nucleic acids, or an expression vector or plurality of expression vectors according to the present disclosure, e.g. by introducing a nucleic acid/plurality of nucleic acids or an expression vector/plurality of expression vectors according to the present disclosure into the at least one immune cell; culturing the modified at least one immune cell specific in in vitro or ex vivo cell culture; generating or expanding a population of modified immune cells; inducing expression of the polypeptide e.g. using a control element and/or a conditional expression system; collecting the modified immune cells; generating or expanding a population of immune cells to be depleted of autoreactive immune cells; contacting a population of immune cells to be depleted of autoreactive immune cells from the subject with the modified at least one immune cell, e.g. in in vitro or ex vivo cell culture; collecting the population of cells depleted of autoreactive immune cells.

Also provided is a method of treating/preventing an autoimmune disease/condition in a subject, the method comprising administering to a subject an immune cell comprising/expressing: (i) an autoantigenic peptide:MHC class I α polypeptide complex and (ii) a polypeptide, a nucleic acid or plurality of nucleic acids, or an expression vector or plurality of expression vectors according to the present disclosure.

Such methods treat/prevent the autoimmune disease/condition by reducing the number of immune cells in the subject specific for the autoantigen, thereby reducing the potential for autoimmunity.

It will be appreciated that the autoimmune disease/condition to be treated by the method corresponds to the autoantigen. That is, where the method is for the treatment/prevention of e.g. rheumatoid arthritis, the autoantigenic peptide is of an autoantigen for which autoreactive T cells are specific in rheumatoid arthritis.

In some embodiments the autoimmune disease/condition to be treated/prevented is selected from: diabetes mellitus type 1, celiac disease, Graves' disease, inflammatory bowel disease, multiple sclerosis, psoriasis, rheumatoid arthritis, and systemic lupus erythematosus.

Where a method is disclosed herein, the present disclosure also provides the articles of the present disclosure for use in such methods. That is to say that the polypeptides, nucleic acids/plurality of nucleic acids, expression vectors/plurality of expression vectors, cells and compositions according to the present disclosure are provided for use in the methods of generating/expanding populations of immune cells, methods of depleting populations of immune cells of alloreactive immune cells, methods of treating/preventing graft rejection following allotransplantation, and methods of treating/preventing a disease/condition by allotransplantation described herein. Also provided is the use of the polypeptides, nucleic acids/plurality of nucleic acids, expression vectors/plurality of expression vectors, cells and compositions according to the present disclosure in the manufacture of products (e.g. medicaments) for use in such methods.

In accordance with embodiments of the present disclosure employing conditional expression systems for inducible expression of the polypeptide of the present disclosure, embodiments of the methods comprise treatment with the appropriate agent for inducing expression of the polypeptide. In some embodiments, treatment may be in vitro or ex vivo, by administration of the agent to an immune cell comprising a nucleic acid/plurality of nucleic acids or an expression vector/plurality of expression vectors according to the present disclosure. In some embodiments, treatment may be in vitro or ex vivo, by administration of the agent to a subject having been administered with an immune cell comprising a nucleic acid/plurality of nucleic acids or an expression vector/plurality of expression vectors according to the present disclosure. In this way, modified immune cells may be stimulated to express the polypeptide of the present disclosure in vitro/ex vivo and/or in vivo.

The skilled person is able to determine appropriate agents and procedures for in accordance with such methods, as appropriate to the conditional expression system. Where a tetracycline-controlled transcriptional activation system is used, the agent may be e.g. tetracycline or doxycycline.

In some embodiments the methods of various aspects of the present disclosure cause less depletion or increased survival of non-alloreactive immune cells as compared to methods employing immunosuppressive agent(s). For example, the present methods are useful for preserving/maintaining the non-alloreactive immune cell compartment in a recipient subject for an allotransplant, or in an allotransplant.

In some embodiments of the methods of the present disclosure comprising allotransplantation, the present methods are associated with an increased number/proportion of non-alloreactive immune cells in the recipient subject for the allotransplant as compared to methods involving treatment with an immunosuppressive agent. In some embodiments of the methods of the present disclosure comprising adoptive transfer of allogeneic immune cells, the present methods are associated with an increased number/proportion of non-alloreactive immune cells in the recipient subject for the allogeneic immune cells as compared to methods involving treatment with an immunosuppressive agent.

In some embodiments of the methods of the present disclosure comprising allotransplantation, the present methods are associated with an increased number/proportion of non-alloreactive immune cells in the allotransplant as compared to methods involving treatment with an immunosuppressive agent.

### Subjects

The subject in accordance with aspects of the present disclosure may be any animal or human. The subject is preferably mammalian, more preferably human. The subject may be a non-human mammal, but is more preferably human. The subject may be male or female. The subject may be a patient. A subject may have been diagnosed with a disease or condition requiring treatment, may be suspected of having such a disease/condition, or may be at risk of developing/contracting such a disease/condition. In some embodiments a subject may be selected for treatment according to the methods of the present disclosure based on characterisation for certain markers of such disease/condition.

### Kits

The present disclosure also provides a kit of parts. In some embodiments the kit may have at least one container having a predetermined quantity of a polypeptide, nucleic acid/plurality of nucleic acids, expression vector/plurality of expression vectors, cell or composition described herein.

The kit may provide the polypeptide, nucleic acid/plurality of nucleic acids, expression vector/plurality of expression vectors, cell or composition together with instructions for use, e.g. for the treatment or prevention of graft rejection or a disease/condition caused or exacerbated by alloreactive immune cells, or to deplete a population of immune cells of alloreactive immune cells (e.g. in the context of treatment/prevention of a disease/condition by adoptive transfer of immune cells).

The kit may additionally instructions for administration to a patient in order to treat/prevent a specified disease/condition. In some embodiments, the kit may comprise materials and/or instructions for producing the polypeptide, nucleic acid/plurality of nucleic acids, expression vector/plurality of expression vectors, cell or composition described herein.

In some embodiments the kit may further comprise at least one container having a predetermined quantity of another therapeutic agent (e.g. anti-infective agent or chemotherapy agent). In such embodiments, the kit may also comprise a second medicament or pharmaceutical composition such that the two medicaments or pharmaceutical compositions may be administered simultaneously or separately such that they provide a combined treatment for the specific disease or condition.

### Sequence Identity

As used herein, “sequence identity” refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using publicly available computer software such as ClustalOmega (Soding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780 software. When using such software, the default parameters, e.g. for gap penalty and extension penalty, are preferably used.

Sequences

| SEQ | | |
|---|---|---|
| ID | | |
| NO: | DESCRIPTION | SEQUENCE |
| 1 | Human B2M | MSRSVALAVLALLSLSGLEAIQRTPKIQVYSRHPAENGKSNFLNCYVSGFHPSDIEVDLLKNGERIE |
| | (UniProt: P61769-1, | KVEHSDLSFSKDWSFYLLYYTEFTPTEKDEYACRVNHVTLSQPKIVKWDRDM |
| | v1) | |
| 2 | Mature human B2M | IQRTPKIQVYSRHPAENGKSNFLNCYVSGFHPSDIEVDLLKNGERIEKVEHSDLSFSKDWSFYLLY |
| | (residues 21-199 of | YTEFTPTEKDEYACRVNHVTLSQPKIVKWDRDM |
| | UniProt: P61769-1, | |
| | v1) | |
| 3 | human B2M Ig-like | PKIQVYSRHPAENGKSNFLNCYVSGFHPSDIEVDLLKNGERIEKVEHSDLSFSKDWSFYLLYYTEF |
| | C1-type domain | TPTEKDEYACRVNHVTLSQPKIV |
| | (residues 25-113 of | |
| | UniProt: P61769-1, | |
| | v1) | |
| 4 | Rhesus macaque | MSRSVALAVLALLSLSGLEAIQRTPKIQVYSRHPPENGKPNFLNCYVSGFHPSDIEVDLLKNGEKM |
| | B2M (UniProt: | GKVEHSDLSFSKDWSFYLLYYTEFTPNEKDEYACRVNHVTLSGPRTVKWDRDM |
| | Q6V7J5-1, v1) | |
| 5 | CD8α | IYIWAPLAGTCGVLLLSLVITLYCNHRN |
| | transmembrane | |
| | domain | |
| 6 | CD28 | WVLVVVGGVLACYSLLVTVAFIIFWV |
| | transmembrane | |
| | domain | |
| 7 | ITAM motif | YXXL/I |
| | | X = any amino acid |
| 8 | ITAM motif | YXXL/I(X)₆₋₈YXXL/I |
| | | X = any amino acid |
| 9 | CD3-ζ | RVKFSRSADAPAYQQGQNQLYNELNLGRREEYDVLDKRRGRDPEMGGKPRRKNPQEGLYNEL |
| | intracellular | QKDKMAEAYSEIGMKGERRRGKGHDGLYQGLSTATKDTYDALHMQALPPR |
| | domain | |
| 10 | CD28 intracellular | RSKRSRLLHSDYMNMTPRRPGPTRKHYQPYAPPRDFAAYRS |
| | domain | |
| 11 | Spacer region | LRWEPSSQPTIPI |
| | (Human HLA-A*02) | |
| 12 | Signal | MSRSVALAVLALLSLSGLEA |
| | peptide (Human | |
| | B2M) | |
| 13 | 2A autocleavage | NPGP |
| | linker sequence | |
| 14 | 2A autocleavage | GSGATNFSLLKQAGDVEENPGP |
| | linker sequence | |
| 15 | Q8 marker | MGLVRRGARAGPRMPRGWTALCLLSLLPSGFMAELPTQGTFSNVSTNVSPAKPTTTPAPRPPTP |
| | | APTIASQPLSLRPEACRPAAGGAVHTRGLDFACDIYIWAPLAGTCGVLLLSLVITLYCNHRNRRRV |
| | | CKCPRPVV |
| 16 | Human B2M coding | ATGAGCAGATCTGTGGCCCTGGCTGTGCTGGCCCTGCTGTCTCTGTCTGGCCTGGAAGCC |
| | sequence | ATCCAGCGGACCCCCAAGATCCAGGTGTACAGCAGACACCCCGCCGAGAACGGCAAGAGC |
| | | AACTTCCTGAACTGCTACGTGTCCGGCTTCCACCCCAGCGACATCGAGGTGGACCTGCTG |
| | | AAGAACGGCGAGCGGATCGAGAAGGTGGAACACAGCGACCTGAGCTTCAGCAAGGACTGG |
| | | TCCTTCTACCTGCTGTACTACACCGAGTTCACCCCCACCGAGAAGGACGAGTACGCCTGC |
| | | AGAGTGAACCACGTGACCCTGAGCCAGCCCAAGATCGTGAAGTGGGACCGGGACATG |
| 17 | Spacer region | CTGAGATGGGAGCCCAGCAGCCAGCCTACCATCCCCATC |
| | (Human HLA-A*02) | |
| | coding sequence | |
| 18 | CD8α | ATCTACATCTGGGCCCCTCTGGCCGGCACCTGTGGCGTGCTGCTGCTGAGTCTCGTGATC |
| | transmembrane | ACCCTGTACTGCAACCACCGGAAC |
| | domain coding | |
| | sequence | |
| 19 | CD3-ζ intracellular | CGAGTGAAGTTTTCCAGAAGTGCGGACGCTCCTGCCTACCAACAAGGCCAGAACCAACTGTA |
| | domain coding | CAATGAGTTAAACTTAGGAAGGAGGGAAGAGTACGACGTGCTAGACAAGCGCCGTGGAAGA |
| | sequence | GATCCTGAAATGGGCGGCAAGCCCAGACGGAAGAACCCCCAGGAAGGCCTGTATAACGAAC |
| | | TGCAGAAAGACAAGATGGCCGAGGCCTACAGCGAGATCGGCATGAAGGGCGAAAGAAGGCG |
| | | GGGCAAGGGCCACGATGGCCTGTATCAGGGACTGAGCACCGCCACCAAGGACACCTACGAC |
| | | GCCCTGCACATGCAGGCTCTGCCTCCGCGG |
| 20 | CD28 | TGGGTGCTGGTTGTGGTGGGAGGGGTACTGGCCTGCTATAGCTTACTGGTGACTGTTGCC |
| | transmembrane | TTTATTATTTTTTGGGTT |
| | domain coding | |
| | sequence | |
| 21 | CD28 intracellular | CGAAGTAAGCGTTCCCGGCTGCTGCACAGTGACTACATGAATATGACCCCAAGACGGCCC |
| | domain coding | GGACCGACAAGGAAACACTATCAACCCTATGCTCCCCCACGAGACTTTGCTGCCTACAGA |
| | sequence | TCA |
| 22 | 2A autocleavage | GGAAGCGGCGCCACAAATTTCAGCCTGCTGAAGCAGGCCGGCGACGTGGAAGAGAATCCC |
| | linker coding | GGCCCT |
| | sequence | |
| 23 | Q8 marker coding | ATGGGACTCGTGCGCAGAGGCGCTAGAGCCGGCCCTAGAATGCCTAGAGGATGGACCGCC |
| | sequence | CTGTGCCTGCTGTCTCTGCTGCCTAGCGGCTTCATGGCCGAGCTGCCTACTCAGGGCACC |
| | | TTCAGCAACGTGTCCACCAATGTGTCCCCAGCGAAGCCCACCACGACGCCAGCGCCGCGA |
| | | CCACCAACACCGGCGCCCACCATCGCGTCGCAGCCCCTGTCCCTGCGCCCAGAGGCGTGC |
| | | CGGCCAGCGGCGGGGGGCGCAGTGCACACGAGGGGGCTGGACTTCGCCTGTGATATCTAC |
| | | ATCTGGGCGCCCTTGGCCGGGACTTGTGGGGTCCTTCTCCTGTCACTGGTTATCACCCTT |
| | | TACTGCAACCACAGGAACCGAAGACGTGTTTGCAAATGTCCCCGGCCTGTGGTC |
| 24 | cFLIP (long isoform) | MSAEVIHQVEEALDTDEKEMLLFLCRDVAIDVVPPNVRDLLDILRERGKLSVGDLAELLYRVRRFDL |
| | | LKRILKMDRKAVETHLLRNPHLVSDYRVLMAEIGEDLDKSDVSSLIFLMKDYMGRGKISKEKSFLDL |
| | | VVELEKLNLVAPDQLDLLEKCLKNIHRIDLKTRIQKYKQSVQGAGTSYRNVLQAAIQKSLKDPSNNF |
| | | RLHNGRSKEQRLKEQLGAQQEPVKKSIQESEAFLPQSIPEERYKMKSKPLGICLIIDCIGNETELLR |
| | | DTFTSLGYEVQKFLHLSMHGISQILGQFACMPEHRDYDSFVCVLVSRGGSQSVYGVDQTHSGLP |
| | | LHHIRRMFMGDSCPYLAGKPKMFFIQNYVVSEGQLEDSSLLEVDGPAMKNVEFKAQKRGLCTVH |
| | | READFFWSLCTADMSLLEQSHSSPSLYLQCLSQKLRQERKRPLLDLHIELNGYMYDWNSRVSAK |
| | | EKYYVWLQHTLRKKLILSYT |
| 25 | PI-9 | METLSNASGTFAIRLLKILCQDNPSHNVFCSPVSISSALAMVLLGAKGNTATQMAQALSLNTEEDIH |
| | | RAFQSLLTEVNKAGTQYLLRTANRLFGEKTCQFLSTFKESCLQFYHAELKELSFIRAAEESRKHINT |
| | | WVSKKTEGKIEELLPGSSIDAETRLVLVNAIYFKGKWNEPFDETYTREMPFKINQEEQRPVQMMY |
| | | QEATFKLAHVGEVRAQLLELPYARKELSLLVLLPDDGVELSTVEKSLTFEKLTAWTKPDCMKSTEV |
| | | EVLLPKFKLQEDYDMESVLRHLGIVDAFQQGKADLSAMSAERDLCLSKFVHKSFVEVNEEGTEAA |
| | | AASSCFVVAESSMESGPRFCADHPFLFFIRHNRANSILFCGRFSSP |
| 26 | cFLIP (long isoform) | ATGAGCGCCGAAGTGATCCACCAGGTGGAAGAGGCCCTGGACACCGACGAGAAAGAGATG |
| | coding sequence | CTGCTGTTCCTGTGCCGCGACGTGGCCATTGATGTGGTGCCTCCAAATGTGCGGGACCTG |
| | | CTGGACATCCTGAGAGAGCGGGGAAAACTGAGCGTGGGAGATCTGGCCGAGCTGCTGTAT |
| | | AGAGTGCGGAGATTCGACCTGCTGAAGCGGATCCTGAAGATGGACCGGAAGGCCGTGGAA |
| | | ACCCATCTGCTGAGAAACCCTCACCTGGTGTCCGACTACAGAGTGCTGATGGCCGAGATC |
| | | GGCGAGGACCTGGATAAGTCCGATGTGTCCAGCCTGATCTTCCTGATGAAGGACTACATG |
| | | GGCAGAGGCAAGATCAGCAAAGAGAAGTCCTTCCTGGACCTGGTGGTGGAACTGGAAAAG |
| | | CTGAACCTGGTGGCCCCTGACCAGCTGGATCTGCTGGAAAAGTGCCTGAAGAACATCCAC |
| | | CGGATCGACCTGAAAACCCGGATCCAAAAGTACAAGCAGAGCGTGCAAGGCGCCGGAACC |
| | | AGCTACAGAAATGTGCTGCAGGCCGCCATCCAGAAGTCCCTGAAGGACCCCAGCAACAAC |
| | | TTCCGGCTGCACAACGGCAGAAGCAAAGAGCAGCGGCTGAAAGAACAGCTGGGAGCCCAG |
| | | CAAGAGCCCGTGAAGAAGTCCATCCAAGAGAGCGAGGCATTCCTGCCTCAGAGCATCCCT |
| | | GAGGAACGGTACAAGATGAAGTCCAAGCCTCTGGGCATCTGCCTGATCATCGACTGCATC |
| | | GGCAACGAGACAGAGCTGCTGAGAGACACCTTTACCAGCCTGGGCTACGAGGTGCAGAAG |
| | | TTCCTGCATCTGAGCATGCACGGCATCAGCCAGATCCTGGGCCAGTTCGCCTGTATGCCC |
| | | GAGCACAGAGACTACGACAGCTTCGTGTGTGTGCTGGTGTCTAGAGGCGGCAGCCAGTCT |
| | | GTGTACGGCGTGGACCAAACACACTCTGGCCTGCCTCTGCACCACATTCGGAGAATGTTC |
| | | ATGGGCGACAGCTGCCCTTATCTGGCCGGCAAGCCCAAGATGTTCTTTATCCAAAACTAC |
| | | GTCGTGTCCGAGGGACAGCTGGAAGATAGCAGCCTGCTGGAAGTGGATGGCCCTGCCATG |
| | | AAGAACGTGGAATTCAAGGCCCAGAAACGGGGCCTGTGTACCGTGCACAGAGAGGCCGAT |
| | | TTCTTCTGGTCACTGTGCACCGCCGACATGTCACTGCTGGAACAGAGCCACTCTAGCCCC |
| | | AGCCTGTACCTGCAGTGTCTGAGCCAGAAGCTGCGGCAAGAGAGAAAGAGGCCCCTGCTC |
| | | GACCTGCACATCGAGCTGAACGGCTATATGTACGACTGGAACAGCCGGGTGTCCGCCAAA |
| | | GAAAAGTACTACGTGTGGCTGCAGCATACCCTGCGGAAGAAGCTGATCCTGTCCTACACC |
| 27 | PI-9 coding | ATGGAAACCCTGAGCAATGCCAGCGGCACCTTCGCCATCAGACTGCTGAAGATCCTGTGC |
| | sequence | CAGGACAACCCCAGCCACAACGTGTTCTGTAGCCCCGTGTCTATCAGCAGCGCCCTGGCT |
| | | ATGGTTCTGCTGGGCGCCAAGGGAAATACCGCCACACAGATGGCTCAGGCCCTGTCTCTG |
| | | AACACCGAAGAGGACATCCACCGGGCCTTTCAGAGCCTGCTGACCGAAGTGAACAAGGCC |
| | | GGCACACAGTACCTGCTGAGAACCGCCAATCGGCTGTTCGGCGAGAAAACCTGCCAGTTC |
| | | CTGAGCACCTTCAAAGAGAGCTGCCTGCAGTTCTACCACGCCGAGCTGAAAGAGCTGAGC |
| | | TTCATCAGAGCCGCCGAGGAAAGCCGGAAGCACATCAATACCTGGGTGTCCAAGAAAACC |
| | | GAGGGCAAGATCGAGGAACTGCTGCCCGGCAGCTCCATCGATGCCGAAACAAGACTGGTG |
| | | CTGGTCAACGCCATCTACTTCAAAGGCAAGTGGAACGAGCCCTTCGACGAGACATACACC |
| | | AGAGAGATGCCCTTCAAGATCAATCAAGAGGAACAGCGGCCCGTGCAGATGATGTACCAA |
| | | GAGGCCACCTTCAAGCTGGCCCATGTGGGAGAAGTTCGGGCCCAACTGCTGGAACTGCCC |
| | | TACGCCAGAAAAGAACTGTCCCTGCTGGTGCTGCTGCCTGACGATGGCGTGGAACTGAGC |
| | | ACCGTGGAAAAGAGCCTGACCTTCGAGAAGCTGACCGCCTGGACCAAGCCTGACTGCATG |
| | | AAGTCCACCGAGGTGGAAGTGCTGCTCCCCAAGTTCAAGCTGCAAGAGGACTACGACATG |
| | | GAAAGCGTGCTGCGGCACCTGGGAATCGTGGATGCTTTCCAGCAGGGCAAAGCCGACCTG |
| | | TCTGCCATGTCTGCCGAGAGGGATCTGTGCCTGAGCAAGTTCGTGCACAAGAGCTTCGTG |
| | | GAAGTGAACGAGGAAGGCACAGAAGCCGCCGCTGCCAGCTCTTGTTTTGTGGTGGCCGAG |
| | | AGCAGCATGGAATCTGGCCCTAGATTCTGCGCCGACCATCCTTTTCTGTTCTTCATCCGG |
| | | CACAACCGGGCCAACAGCATCCTGTTCTGTGGCAGATTCAGCAGCCCC |

The present disclosure includes the combination of the aspects and preferred features described except where such a combination is clearly impermissible or expressly avoided.

The section headings used herein are for organizational purposes only and are not to be construed as limiting the subject matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Where a nucleic acid sequence is disclosed herein, the reverse complement thereof is also expressly contemplated.

Methods described herein may preferably performed in vitro. The term “in vitro” is intended to encompass procedures performed with cells in culture whereas the term “in vivo” is intended to encompass procedures with/on intact multi-cellular organisms.

### BRIEF DESCRIPTION OF THE FIGURES

Embodiments and experiments illustrating the principles of the present disclosure will now be discussed with reference to the accompanying figures.

FIG. 1. Scatterplots showing expression of HLA-A2 and CD3 of the population of cells obtained following the indicated number of days in coculture comprising recipient PBMCs and non-transduced 3^{rd} party VSTs (left panels) or CHAR-expressing 3^{rd} party VSTs (right panels).

FIG. 2. Scatterplots showing expression of CHAR construct by transduced cells (right panel), but not non-transduced cells (left panel).

FIG. 3. Graph showing fold expansion of non-transduced (NT) VSTs and CHAR-construct transduced (CHAR) VSTs in culture at the indicated number of days after transduction.

FIGS. 4A and 4B. Schematic representation of an inducible CHAR construct (4A), and scatterplots showing expression of the construct shown in FIG. 4A by cells transduced with the construct (4B), in the absence of doxycycline (left panel), or in the presence of doxycycline (right panel). The construct employs a Tet-On 3G conditional expression system; the CHAR construct is only expressed in the presence of doxycycline.

FIGS. 5A and 5B. Schematic representation of an inducible CHAR (iCHAR) construct (5A), and scatterplots showing expression of the construct shown in FIG. 5A by cells transduced with the construct (5B) in the absence of doxycycline (left panel), or in the presence of doxycycline (right panel).

FIG. 6. Graph showing fold expansion in culture of non-transduced (NT) VSTs and VSTs transduced with the construct shown in FIG. 5A at the indicated number of days after transduction, in the absence of doxycycline.

FIG. 7. Scatterplots showing expression of HLA-A2 and CD3 of the population of cells obtained following the indicated number of days in coculture comprising recipient PBMCs and non-transduced 3^{rd} party VSTs (left panels) or iCHAR-expressing 3rd party VSTs (right panels).

FIG. 8. Scatterplots showing expression of HLA-A2 and CD3 in cells obtained following the indicated number of days in coculture comprising recipient PBMCs and non-transduced 3^{rd} party VSTs (left panels), iCHAR-expressing 3^{rd} party VSTs (middle panels), or iCHAR-CD28 expressing 3^{rd} party EBVSTs additionally transduced with factors capable of inhibiting apoptosis: cFLIP and PI-9 (right panels).

### EXAMPLES

In the following Examples, the inventors describe the production and characterisation of chimeric HLA accessory receptor molecules, constructs encoding and cells expressing the same.

The inventors provide a proof of concept that primary human T cells can be engineered to recognise and target mismatched alloreactive T cells, which can cause severe complications for allogeneic transplant recipients.

### Example 1: Design of a Chimeric HLA Accessory Receptor (CHAR), and Generation of CHAR-Expressing Cells

The inventors prepared nucleic acid construct encoding a chimeric HLA accessory receptor (CHAR), a fusion polypeptide having the following relative arrangement of amino acid sequences:

(N-term) [signal sequence]-[human B2M]-[spacer region]-[human CD8α transmembrane domain]-[human CD3ζ intracellular domain]-[2A autocleavage sequence]-[Q8] (C-term)

As explained hereinabove, “Q8” is a detectable marker comprising a 16 amino acid epitope of CD34 fused to stalk region of CD8a, and is described e.g. in Philip et al., Blood (2014) 124:1277-1287 (incorporated by reference herein). The amino acid sequence for Q8 is shown in SEQ ID NO:15, and the nucleic acid sequence encoding Q8 is shown in SEQ ID NO:23.

The construct was cloned into a SFG retroviral vector backbone and retrovirus was generated pseudotyped with an RD114 envelope. Transduction was performed by centrifuging retroviral supernatants in wells of cell culture plates coated with RetroNectin reagent (Clontech), removing the retroviral supernatants, and subsequently applying the cells to be transduced to the wells of the plates.

The construct was first expressed in Daudi cells, which lack expression of surface HLA, due to lack of endogenous B2M. Expression of the CHAR molecule was found to restore expression of HLA to the cell surface of the Daudi cells, indicating that the CHAR molecule was able to form a complex with endogenously-expressed HLA class I α molecules.

The construct was also transduced into VSTs. VSTs were initiated by stimulation of PBMCs with pepmixes spanning viral antigens. VSTs were grown in cell culture media containing human IL-7 at 10 ng/ml and human IL-15 at 10 ng/ml, and were transduced with CHAR construct at 4-5 days after initiation of the VSTs. The VSTs were subsequently restimulated on day 9 with irradiated autologous activated T cells (AATCs) pulsed with the same pepmixes as those used in the initial stimulation, in the presence of aK562 cells expressing co-stimulatory molecules CD80, CD83, CD86 and 4-1 BB ligand (K562-cs), as described in US 20150017723 A1

### Example 2: Ability of CHAR to Protect Allogeneic VSTs from Elimination by Alloreactive T Cells

The inventors investigated the effect of CHAR expression on rejection of allogeneic VSTs in vitro.

Briefly, a population of 1×10⁶ PBMCs from a subject (recipient) was co-cultured in a mixed lymphocyte reaction (MLR) assay with (i) 0.5×10⁶ VSTs generated from PBMCs of another subject (3^{rd} party) having a different HLA type to the HLA type of subject 1, or (ii) 0.5×10⁶ VSTs generated from PBMCs of the 3rd party, additionally transduced with construct encoding the CHAR. Human IL-2 was added to the MLR assay at 20 IU/ml.

Flow cytometric analysis was performed after the indicated number of days, and absolute cell numbers were determined using counting beads. The Gallios Flow Cytometer (Beckman Coulter) was used to acquire events, and Kaluza Analysis Software (Beckman Coulter) was used for data analysis and graphical representation.

When CHAR-VSTs encountered T cells that recognised them as targets, they became activated and degranulated. T cells derived from the recipient or 3rd party could be identified in the population obtained following coculture based on expression of HLA-A2; recipient's cells expressed HLA-A2, whereas 3^{rd} party's cells did not.

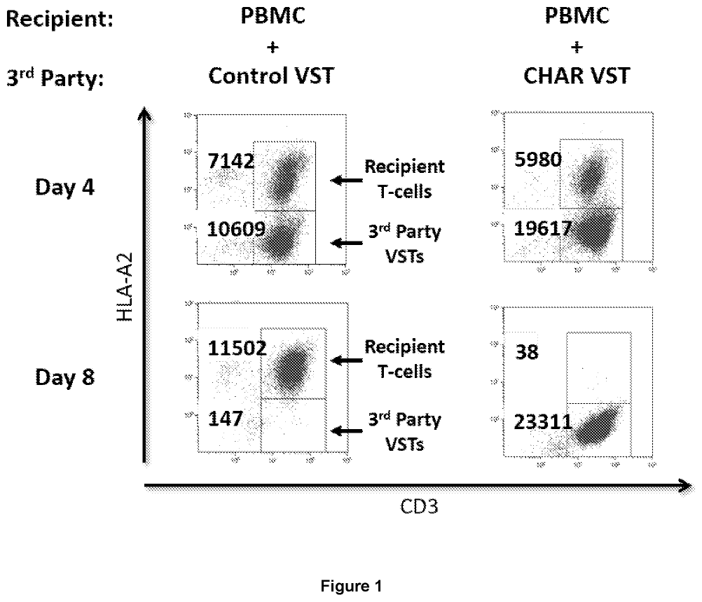

The results are shown in FIG. 1. As expected, co-culture of recipient PBMCs with non-transduced 3^{rd} party VSTs resulted in elimination of the 3rd Party VSTs after 8 days (FIG. 1, left panels). By contrast, 3^{rd} party VSTs expressing CHAR persisted in cocultures at day 8 (FIG. 1, right panels).

The inventors confirmed the loss of alloreactive T cells from the recipient PBMCs in a secondary MLR by restimulating them with PBMC autologous to the CHAR-VSTs. The lack of proliferation observed confirmed that T cells reactive to the HLA antigens of the CHAR-VSTs had been eliminated.

The inventors also confirmed that non-alloreactive T cells (the majority of T cells within PBMCs) were retained after exposure to CHAR T cells, by measuring the frequency and function of virus-specific T cells within the PBMC population after culturing with CHAR T cells.

Thus the CHAR was shown to be able to protect allogeneic VSTs from elimination by alloreactive T cells from within the recipient PBMC population.

The inventors further observed that the CHAR T cells were able to eliminate resting alloreactive T cells within the population of PBMCs, but not pre-activated alloreactive T cells (in co-cultures with pre-activated alloreactive T cells, CHAR-T cells were eliminated).

The results suggest that CHAR-expressing T cells could be particularly useful in clinical settings in which alloreactive T cells would be resting upon first encounter with CHAR T cells, such as in living kidney transplantation, where CHART cells derived from the kidney donor could be infused into a recipient prior to transplant to eliminate alloreactive T cells that could otherwise result in graft rejection.

Alternatively, in the case of hematopoietic stem cell transfer (HSCT), CHAR-expressing recipient T cells could be cultured with stem cell grafts prior to infusion, in order to eliminate alloreactive T cells in the stem cell graft that could otherwise attack host tissues.

### Example 3: Analysis of Proliferation of Cells Expressing the CHAR

Cells were analysed by flow cytometry for expression of the construct by analysis using an anti-CD34 antibody capable of recognising Q8. The Gallios flow cytometer (Beckman Coulter) was used to acquire events and Kaluza Analysis Software (Beckman Coulter) was used for data analysis and graphical representation.

Q8 served as a marker for successfully transduced cells expressing the construct. Fold expansion of cells expressing the CHAR was monitored over time and compared to the fold expansion of non-transduced cells.

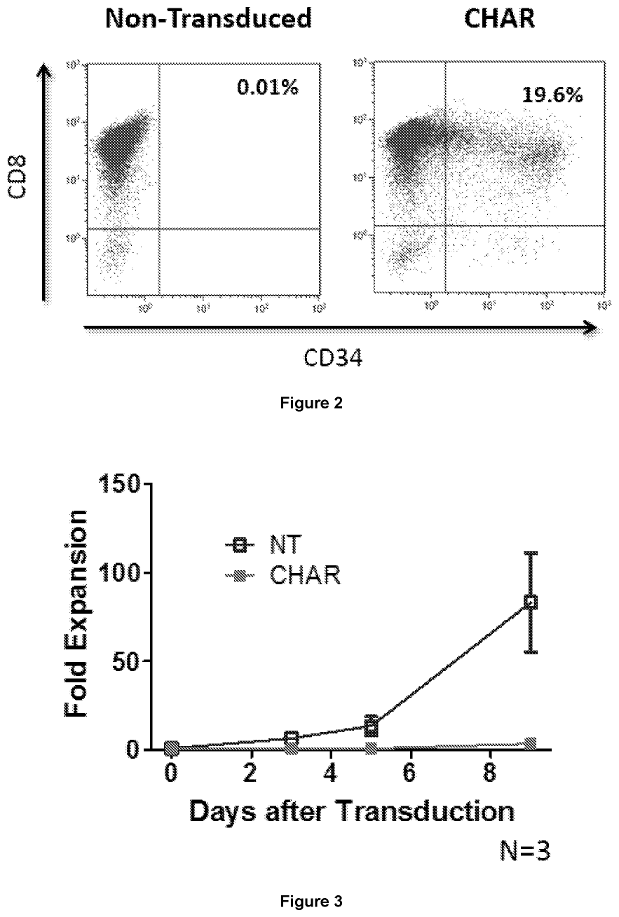

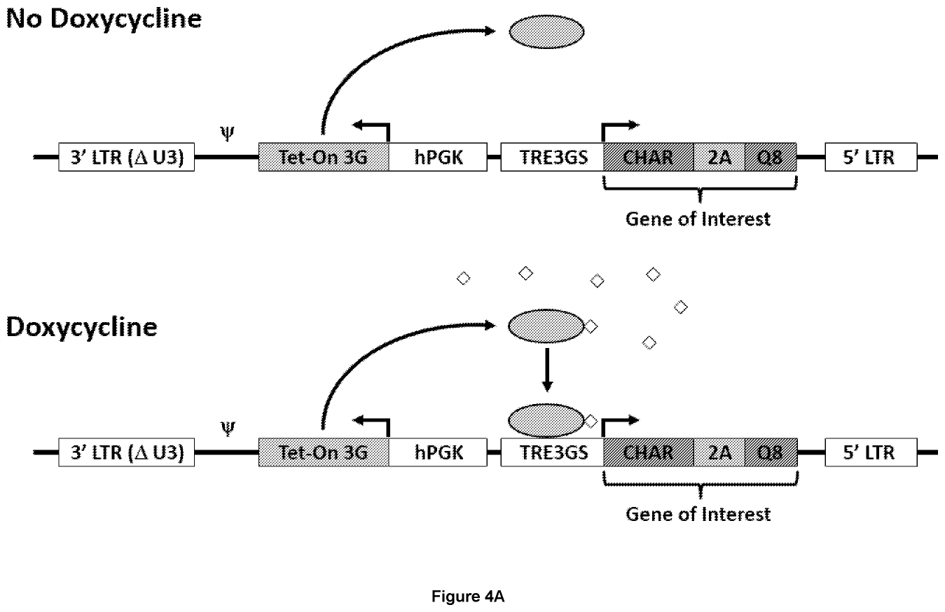

The results are shown in FIGS. 2 and 3; CHAR-expressing VSTs were found to proliferate less than their non-transduced counterparts, possibly due to fratricide.

### Example 4: Design and Characterisation of an Inducible CHAR

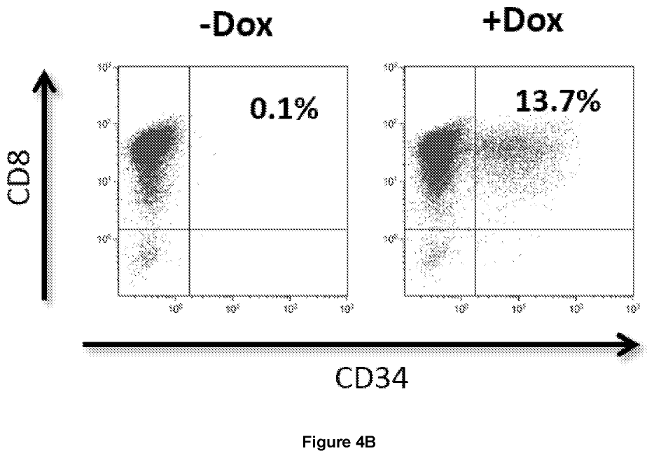

The inventors next designed a construct encoding an inducible CHAR. Briefly, nucleic acid encoding the CHAR construct (see Example 1) was cloned into a Tet-On 3G system providing for inducible expression of nucleic acid encoding the CHAR in the presence of doxycycline (see schematic of FIG. 4A).

VSTs were transduced with retroviral vector encoding the inducible CHAR construct, and subsequently analysed for expression of the CHAR in the presence or absence of doxycycline treatment by analysis of Q8 expression as determined using anti-CD34 antibody. Doxcycline was added at 100 ng/ml to VSTs for 24 hrs, and then CD34 expression was analysed by flow cytometry to determine expression of the CHAR construct.

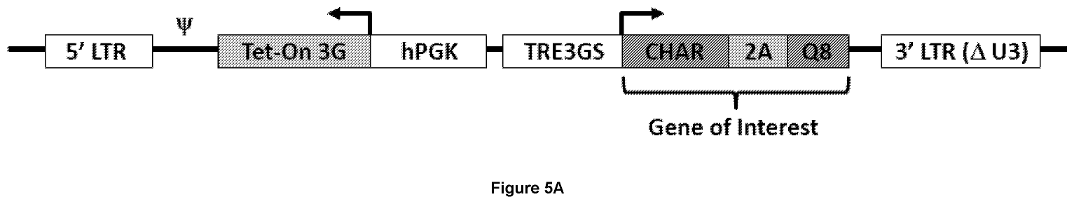

Doxcycline was found to induce expression of the construct. However, the level of transduction was observed to be low (see FIG. 4B). Modification of the construct to invert the entire inducible unit between the 5′ LTR and 3′ LTR (FIG. 5A) was found to improve transduction efficiency (FIG. 5B).

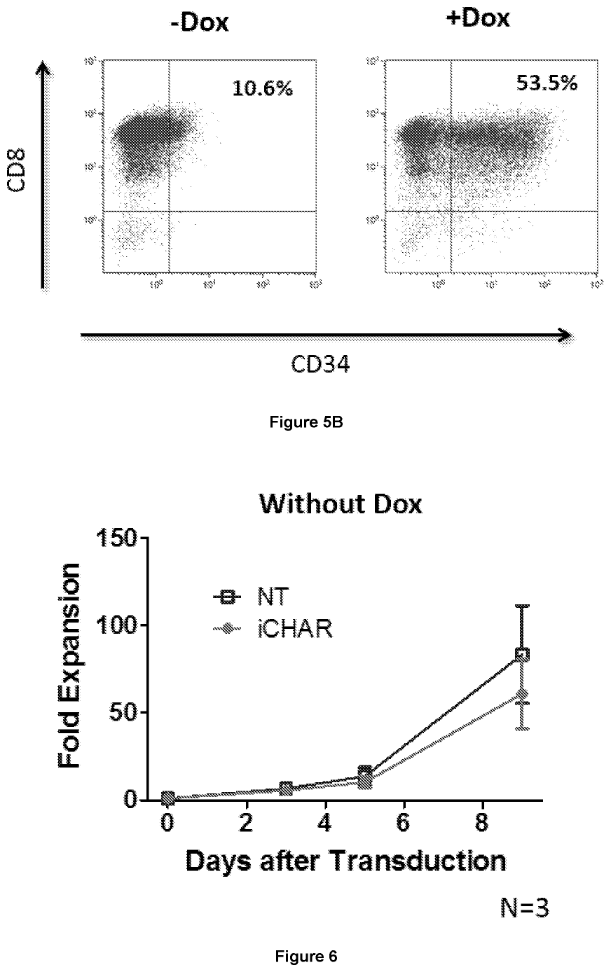

VSTs expressing the iCHAR construct shown in FIG. 5A displayed expansion similar to non-transduced cells in the absence of doxycycline (FIG. 6).

### Example 5: Ability of iCHAR to Protect Allogeneic VSTs from Elimination by Alloreactive T Cells

The inventors investigated the effect of iCHAR expression on rejection of allogeneic VSTs in vitro.

Briefly, a population of 1×10⁶ PBMCs from a subject (recipient) was co-cultured in a mixed lymphocyte reaction (MLR) assay with (i) 0.5×10⁶ VSTs generated from PBMCs of another subject (3^{rd} party) having a different HLA type to the HLA type of subject 1 (non-transduced VSTs), or (ii) 0.5×10⁶ VSTs generated from PBMCs of the 3^{rd} party, additionally transduced with construct encoding the iCHAR construct shown in FIG. 5A (see Example 4). The cocultures were performed in the presence of 100 ng/ml doxycycline for expression of the iCHAR. Human IL-2 was added to the MLR assay at 20 IU/ml.

Flow cytometric analysis was performed after the indicated number of days, and absolute cell numbers were determined using counting beads. The Gallios Flow Cytometer (Beckman Coulter) was used to acquire events, and Kaluza Analysis Software (Beckman Coulter) was used for data analysis and graphical representation.

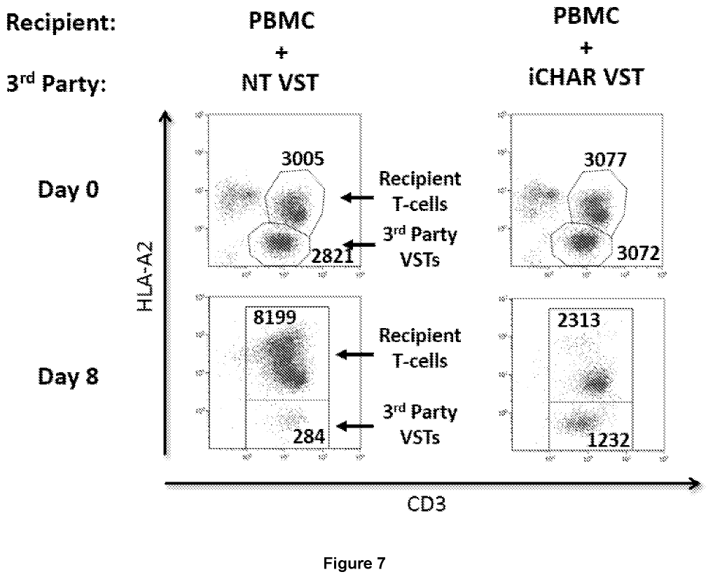

The results are shown in FIG. 7. As expected, co-culture of recipient PBMCs with non-transduced 3^{rd} party VSTs resulted in elimination of the 3rd Party VSTs after 8 days (FIG. 7, left panels). By contrast, 3^{rd} party VSTs expressing iCHAR persisted in cocultures at day 8 (FIG. 7, right panels).

### Example 6: Design of a CHAR and CHAR-Expressing Cells Having Improved Survival and Persistence

The inventors prepared retroviral vector encoding a chimeric HLA accessory receptor (CHAR) having the following relative arrangement of amino acid sequences:

(N-term) [human B2M]-[spacer region]-[human CD28 transmembrane domain]-[human CD28 costimulatory domain]-[human CD3 intracellular domain] (C-term)

The construct employed a Tet-On 3G system for inducible expression of the CHAR.

The inventors also constructed a retroviral vector encoding factors capable of inhibiting apoptosis, specifically the death receptor inhibitor cFLIP variant having the amino acid sequence shown in SEQ ID NO:24, and the granzyme B inhibitor PI-9 variant having the amino acid sequence shown in SEQ ID NO:25.

Retroviruses were generated for the inducible CHAR containing the CD28 costimulatory domain (iCHAR-CD28), and also for cFLIP and PI-9. For co-transductions, retroviruses were mixed together and centrifuged in wells of cell culture plates coated with RetroNectin. VSTs were transduced on Days 4-5 and restimulated on Day 9.

The inventors investigated the effect of iCHAR-CD28, cFLIP and PI-9 on the survival and persistence of VSTs co-cultured with allogeneic PBMCs.

Briefly, a population of 1×10⁶ PBMCs from a subject (recipient) was co-cultured in a mixed lymphocyte reaction (MLR) assay with (i) 0.5×10⁶ VSTs generated from PBMCs of another subject (3rd party) having a different HLA type to the HLA type of subject 1 (non-transduced VSTs), (ii) 0.5×10⁶ VSTs generated from PBMCs of the 3rd party, additionally transduced with the iCHAR construct shown in FIG. 5A, or (iii) 0.5×10⁶ VSTs generated from PBMCs of the 3^{rd} party, additionally transduced with constructs encoding iCHAR-CD28, cFLIP and PI-9.

The cocultures were performed in the presence of 100 ng/ml doxycycline for expression of the CHAR constructs. Human IL-2 was added to the the MLR at 20 IU/ml. Flow cytometric analysis was performed after the indicated number of days, and absolute cell numbers were determined using counting beads. The Gallios Flow Cytometer (Beckman Coulter) was used to acquire events, and Kaluza Analysis Software (Beckman Coulter) was used for data analysis and graphical representation.

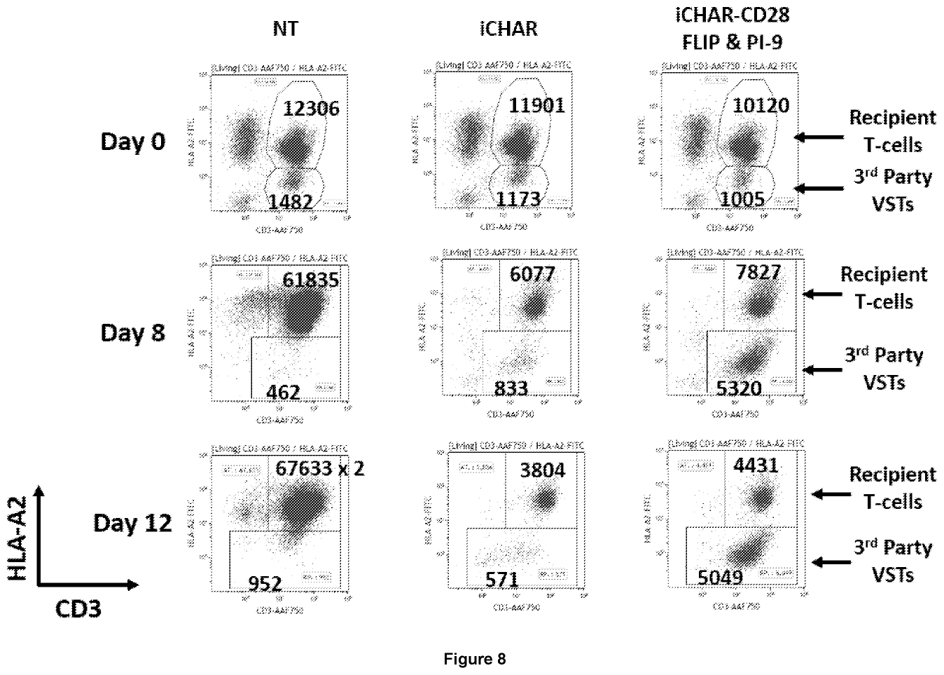

The results are shown in FIG. 8. VSTs expressing an iCHAR with a CD28 costimulatory domain additionally transduced to increase expression of cFLIP and PI-9 were shown to have increased survival in co-culture with allogeneic PBMCs as compared to iCHAR-expressing VSTs which were not modified to express cFLIP and PI-9.

1. A polypeptide, optionally isolated, comprising: (i) an MHC class I α polypeptide association domain, (ii) a transmembrane domain, and (iii) a signalling domain comprising an ITAM-containing sequence.

2. The polypeptide according to claim 1, wherein the MHC class I α polypeptide association domain comprises an amino acid sequence which is, or which is derived from, the Ig-like C1-type domain of B2M.

3. The polypeptide according to claim 1 or claim 2, wherein the MHC class I α polypeptide association domain comprises an amino acid sequence having at least 70% amino acid sequence identity to SEQ ID NO:3.

4. The polypeptide according to any one of claims 1 to 3, wherein the signalling domain comprises an amino acid sequence which is, or which is derived from, the intracellular domain of CD3-ζ.

5. The polypeptide according to any one of claims 1 to 4, wherein the signalling domain comprises an amino acid sequence having at least 70% amino acid sequence identity to SEQ ID NO:9.

6. The polypeptide according to any one of claims 1 to 5, wherein the transmembrane domain comprises an amino acid sequence which is, or which is derived from, the transmembrane domain of CD8α or CD28.

7. The polypeptide according to any one of claims 1 to 6, wherein the transmembrane domain comprises an amino acid sequence having at least 70% amino acid sequence identity to SEQ ID NO:5 or SEQ ID NO:6.

8. The polypeptide according to any one of claims 1 to 7, wherein the signalling domain additionally comprises a costimulatory sequence.

9. The polypeptide according to claim 8, wherein the costimulatory sequence is, or is derived from, the intracellular domain of CD28.

10. The polypeptide according to any one of claims 1 to 9, wherein the signalling domain comprises an amino acid sequence having at least 70% amino acid sequence identity to SEQ ID NO:10.

11. The polypeptide according to any one of claims 1 to 10, wherein the polypeptide additionally comprises a spacer region between the MHC class I α polypeptide association domain and the transmembrane domain.

12. The polypeptide according to claim 11, wherein the spacer region comprises an amino acid sequence having at least 70% amino acid sequence identity to SEQ ID NO:11.

13. A nucleic acid, or a plurality of nucleic acids, optionally isolated, encoding a polypeptide according to any one of claims 1 to 12.

14. The nucleic acid or plurality of nucleic acids according to claim 13, comprising a control element for inducible upregulation of expression of the polypeptide.

15. The nucleic acid or plurality of nucleic acids according to claim 13 or claim 14, wherein the nucleic acid or plurality of nucleic acids encodes a conditional expression system for controlling expression of the polypeptide.

16. The nucleic acid or plurality of nucleic acids according to claim 15, wherein the conditional expression system for controlling expression of the polypeptide is a Tet-On system.

17. An expression vector, or a plurality of expression vectors, comprising a nucleic acid or a plurality of nucleic acids according to any one of claims 13 to 16.

18. A cell comprising a polypeptide according to any one of claims 1 to 12, a nucleic acid or plurality of nucleic acids according to any one of claims 13 to 16, or an expression vector or plurality of expression vectors according to claim 17.

19. The cell according to claim 18, wherein the cell is virus-specific T cell.

20. A method comprising culturing a cell comprising a nucleic acid or a plurality of nucleic acids according to any one of claims 13 to 16, or an expression vector or a plurality of expression vectors according to claim 17, under conditions suitable for expression of the polypeptide from the nucleic acid(s) or expression vector(s).

21. A method of generating or expanding a population of immune cells, comprising modifying an immune cell to express or comprise a polypeptide according to any one of claims 1 to 12, a nucleic acid or plurality of nucleic acids according to any one of claims 13 to 16, or an expression vector or plurality of expression vectors according to claim 17.

22. A method of generating or expanding a population of immune cells, comprising:
(a) isolating immune cells from a subject;
(b) modifying at least one immune cell to express or comprise a polypeptide according to any one of claims 1 to 12, a nucleic acid or plurality of nucleic acids according to any one of claims 13 to 16, or an expression vector or plurality of expression vectors according to claim 17; and
(c) optionally expanding the modified at least one immune cell.

23. A method of generating or expanding a population of virus-specific immune cells, comprising:
(a) isolating immune cells from a subject;
(b) generating or expanding a population of virus-specific immune cells by a method comprising: stimulating the immune cells by culture in the presence of antigen presenting cells (APCs) presenting a peptide of the virus;
(c) modifying at least one virus-specific immune cell to express or comprise a polypeptide according to any one of claims 1 to 12, a nucleic acid or plurality of nucleic acids according to any one of claims 13 to 16, or an expression vector or plurality of expression vectors according to claim 17; and
(d) optionally expanding the modified at least one virus-specific immune cell.

24. A cell obtained or obtainable by a method according to any one of claims 21 to 23.

25. The cell according to any one of claim 18, 19 or 24, additionally comprising modification to increase expression/activity of one or more factors capable of inhibiting apoptosis.

26. A composition comprising a polypeptide according to any one of claims 1 to 12, a nucleic acid or a plurality of nucleic acids according to any one of claims 13 to 16, an expression vector or a plurality of expression vectors according to claim 17, or a cell according to any one of claim 18, 19, 24 or 25.

27. A nucleic acid or a plurality of nucleic acids according to any one of claims 13 to 16, an expression vector or a plurality of expression vectors according to claim 17, a cell according to any one of claim 18, 19, 24 or 25, or a composition according to claim 26 for use in a method of medical treatment or prophylaxis.

28. A method of depleting a population of immune cells of alloreactive immune cells, comprising:
(a) modifying at least one immune cell from a first subject to express or comprise a polypeptide according to any one of claims 1 to 12, a nucleic acid or plurality of nucleic acids according to any one of claims 13 to 16, or an expression vector or plurality of expression vectors according to claim 17; and
(b) contacting a population of immune cells to be depleted of alloreactive immune cells from a second, allogeneic subject with the modified at least one immune cell.

29. A method of treating/preventing graft rejection following allotransplantation, comprising administering at least one immune cell of the donor subject for the allotransplant modified to express or comprise a polypeptide according to any one of claims 1 to 12, a nucleic acid or plurality of nucleic acids according to any one of claims 13 to 16, or an expression vector or plurality of expression vectors according to claim 17 to the recipient subject for the allotransplant.

30. A method of treating/preventing graft versus host disease (GVHD) associated with allotransplantation, comprising contacting the allotransplant with at least one immune cell of the recipient subject for the allotransplant modified to express or comprise a polypeptide according to any one of claims 1 to 12, a nucleic acid or plurality of nucleic acids according to any one of claims 13 to 16, or an expression vector or plurality of expression vectors according to claim 17.

31. A method of treating/preventing a disease/condition by allotransplantation, comprising:
(a) modifying at least one immune cell from the donor subject to express or comprise a polypeptide according to any one of claims 1 to 12, a nucleic acid or plurality of nucleic acids according to any one of claims 13 to 16, or an expression vector or plurality of expression vectors according to claim 17; and
(b) administering the modified at least one immune cell to the recipient subject for the allotransplant.

32. A method of treating/preventing a disease/condition by allotransplantation, comprising:
(a) modifying at least one immune cell from the recipient subject for the allotransplant to express or comprise a polypeptide according to any one of claims 1 to 12, a nucleic acid or plurality of nucleic acids according to any one of claims 13 to 16, or an expression vector or plurality of expression vectors according to claim 17; and
(b) contacting the allotransplant with the modified at least one immune cell.

33. The method according to claim 31 or claim 32, wherein the allotransplantation comprises adoptive transfer of allogeneic immune cells.

34. A method of treating/preventing a disease/condition by adoptive transfer of allogeneic immune cells, comprising:
(a) isolating immune cells from a subject;
(b) modifying at least one immune cell to express or comprise a polypeptide according to any one of claims 1 to 12, a nucleic acid or plurality of nucleic acids according to any one of claims 13 to 16, or an expression vector or plurality of expression vectors according to claim 17;
(c) optionally expanding the modified at least one immune cell, and;
(d) administering the modified at least one immune cell to a subject.

35. A method of treating/preventing a disease/condition by adoptive transfer of allogeneic immune cells specific for a virus, comprising:
(a) isolating immune cells from a subject;
(b) generating or expanding a population of immune cells specific for a virus by a method comprising: stimulating the immune cells by culture in the presence of antigen presenting cells (APCs) presenting a peptide of the virus;
(c) modifying at least one immune cell specific for a virus to express or comprise a polypeptide according to any one of claims 1 to 12, a nucleic acid or plurality of nucleic acids according to any one of claims 13 to 16, or an expression vector or plurality of expression vectors according to claim 17;
(d) optionally expanding the modified at least one immune cell specific for a virus, and;
(e) administering the modified at least one immune cell specific for a virus to a subject.

36. The method according to any one of claims 31 to 35, wherein the disease/condition is a T cell dysfunctional disorder, a cancer or an infectious disease.

37. The method according to claim 36, wherein the cancer is selected from the group consisting of: colon cancer, colon carcinoma, colorectal cancer, nasopharyngeal carcinoma, cervical carcinoma, oropharyngeal carcinoma, gastric carcinoma, hepatocellular carcinoma, head and neck cancer, head and neck squamous cell carcinoma (HNSCC), oral cancer, laryngeal cancer, prostate cancer, lung cancer, small cell lung cancer, non-small cell lung cancer, bladder cancer, urothelial carcinoma, melanoma, advanced melanoma, renal cell carcinoma, ovarian cancer or mesothelioma.

38. A method of depleting a population of immune cells of autoreactive immune cells, comprising:
(a) modifying at least one immune cell comprising/expressing an autoantigenic peptide:MHC class I α polypeptide complex to express or comprise a polypeptide according to any one of claims 1 to 12, a nucleic acid or plurality of nucleic acids according to any one of claims 13 to 16, or an expression vector or plurality of expression vectors according to claim 17; and
(b) contacting a population of immune cells to be depleted of autoreactive immune cells (e.g. autoreactive T cells) with the modified at least one immune cell.

39. A method of treating/preventing an autoimmune disease/condition in a subject, the method comprising administering to a subject an immune cell comprising/expressing: (i) an autoantigenic peptide:MHC class I α polypeptide complex and (ii) a polypeptide according to any one of claims 1 to 12, a nucleic acid or plurality of nucleic acids according to any one of claims 13 to 16, or an expression vector or plurality of expression vectors according to claim 17.
